(19) United States
(12) Patent Application Publication
(10) Pub. No.: US 2026/0120849 A1
(43) Pub. Date: Apr. 30, 2026

(54) METHOD AND DEVICE FOR PROCESSING IMAGES OF MEDICAL CONDITIONS

(71) Applicant: The Hong Kong University of Science and Technology, Hong Kong (CN)

(72) Inventors: Hao CHEN, Hong Kong (CN); Sunan HE, Hong Kong (CN); Yuxiang NIE, Hong Kong (CN)

(21) Appl. No.: 19/283,478

(22) Filed: Jul. 29, 2025

Related U.S. Application Data
(60) Provisional application No. 63/714,144, filed on Oct. 31, 2024.

(51) Int. Cl. G16H 30/40; G16H 10/60; G16H 15/00; G16H 50/20

(52) U.S. Cl. CPC G16H 30/40; G16H 10/60; G16H 15/00; G16H 50/20

(57) ABSTRACT

A computer-implemented method (300) for processing a sample image of a medical condition is disclosed, which comprises: processing (305), by a set of computer vision models for a medical modality associated with the medical condition, the sample image to obtain a set of determination related to a plurality of predicted diagnoses for the medical condition; processing (310) the sample image to determine an associated visual embedding to be used as a query for querying a database; querying (315), based on vector similarity between the determined visual embedding and the respective keys in the plurality of the entries, the database to retrieve a top-k most similar entries from the database; and processing (320), by a medically-trained generalist foundation model (GFM) using the set of determination as reference context and images corresponding with the retrieved top-k most similar entries, the sample image to make a predicted determination on the medical condition shown by the sample image.

### CROSS REFERENCE TO RELATED APPLICATIONS

The present application claims priority to Provisional Application No. 63/714,144 filed in the U.S. Patent and Trademark Office on Oct. 31, 2024, the entire contents of which are incorporated herein by reference.

### FIELD OF INVENTION

The following relates generally to image processing, and more specifically, it relates to a computer-implemented method for processing sample images of medical conditions or diseases.

### BACKGROUND

Advanced by the rapid development of Large Language Models (LLMs) [ref. 1-3] as well as vision-language pre-training [ref. 4-6], large-scale vision-language models (LVLMs) [ref. 7-9] have demonstrated remarkable performance in a wide range of tasks (e.g., visual question answering [ref. 10] and image captioning [ref. 11]), thus establishing themselves as generalist foundation models (GFMs).

In the realm of medicine/healthcare, GFMs [ref. 12-18] also showcased impressive proficiency in generalizing across various tasks, such as visual question answering [ref. 19-20] and radiology report generation tasks [ref. 21-24]. The remarkable generalizability of GFMs can be attributed to two key aspects. First, the extensive and diverse training corpus endows the models with comprehensive medical knowledge. Additionally, the powerful instruction following and in-context learning abilities of GFMs improve their versatility and flexibility, facilitating their applications across a multitude of tasks.

While GFMs seem to exhibit superior generalizability, specialist models however excel in precision-based tasks. Tailored for specific downstream tasks, these specialist models possess profound domain-specific knowledge, which enable them to concentrate on a narrower scope and deliver more precise results for analysing images. For instance, in medical image diagnosis tasks [ref. 25-27], specialist models surpass the GFMs and demonstrate superior performance [ref. 15, 28-29]. Hence, GFMs are characterised by generalizability and flexibility, whereas specialist models possess specialist expertise and precision.

Hence, there exists a need for a solution that may address at least one of the problems of the prior art, and/or to provide a choice that is useful in the art.

### SUMMARY

The described techniques herein may relate to a method and device for processing sample images of medical conditions or diseases.

According to a 1^{st} aspect, there is disclosed a computer-implemented method for training a generalist foundation model (GFM), the method comprises: generating, by the GFM to be provided as a first dataset, a plurality of medical reports related to different medical conditions, wherein each medical report is generated based on an associated image showing a medical condition, and classification labels that include textual description about the medical condition, and a medical modality associated with the medical condition; generating, by the GFM, textual description for respective images showing different medical conditions, wherein the textual description describe the medical conditions, based on diagnostically relevant visual features shown in the respective images, and wherein the generated textual description and the respective images are provided as a second dataset; configuring a training dataset to include the first dataset, the second dataset, a third dataset associated with Medical Image Diagnosis (CLS), a fourth dataset associated with Medical Report Generation (MRG), and a fifth dataset associated with Visual Question Answering (VQA); and training, based on the training dataset, the GFM to obtain a medically-trained GFM.

Additionally or alternatively, the GFM may include being selected from one of: RadFM, LLaVA-Med, Med-Flamingo, MedDr, and Intern VL.

Additionally or alternatively, each medical report may be configured to include, based on processing by the GFM, information of medical findings and impressions about the medical condition shown by the associated image.

Additionally or alternatively, the respective images showing the different medical conditions may be obtained from OpenI.

Additionally or alternatively, the medical modality may include one of: radiology, pathology, dermatology, ophthalmology, gastroenterology, fundoscopy, chest X-ray, and endoscopy.

According to a 2^{nd} aspect, there is disclosed a computer-implemented method for processing a sample image of a medical condition, the method comprises: processing, by a set of computer vision models for a medical modality associated with the medical condition, the sample image to obtain a set of determination related to a plurality of predicted diagnoses for the medical condition, wherein the set of computer vision models are selected from a plurality of different sets of computer vision models, wherein respective sets of computer vision models are pretrained with downstream datasets specific to respective medical modalities for different medical conditions; processing, by an embedding model, the sample image to determine an associated visual embedding to be used as a query for querying a database, wherein the database include a plurality of entries of value-key pairs associated with respective images of different medical conditions; and wherein a key represents a visual embedding of an image, and an associated value provides textual description of the medical condition shown in said image; querying, by at least one computer vision model in the selected set of computer vision models based on vector similarity between the determined visual embedding and the respective keys in the plurality of the entries, the database to retrieve a top-k most similar entries from the database, wherein k is a positive integer; and processing, by a medically-trained generalist foundation model (GFM) using the obtained set of determination as reference context and images corresponding with the retrieved top-k most similar entries, the sample image to make a predicted determination on the medical condition shown by the sample image, wherein the medically-trained GFM is trained in accordance with the method of claim 1.

Additionally or alternatively, the vector similarity may be performed based on cosine similarity.

According to a 3^{rd} aspect, there is disclosed a computing device for training a generalist foundation model (GFM), comprising: one or more memories having executable code; and one or more processors coupled to the one or more memories, and configured to execute the code to cause the device to: generate, by the GFM to be provided as a first dataset, a plurality of medical reports related to different medical conditions, wherein each medical report is generated based on an associated image showing a medical condition, and classification labels that include textual description about the medical condition, and a medical modality associated with the medical condition; generate, by the GFM, textual description for respective images showing different medical conditions, wherein the textual description describe the medical conditions, based on diagnostically relevant visual features shown in the respective images, and wherein the generated textual description and the respective images are provided as a second dataset; configure a training dataset to include the first dataset, the second dataset, a third dataset associated with Medical Image Diagnosis (CLS), a fourth dataset associated with Medical Report Generation (MRG), and a fifth dataset associated with Visual Question Answering (VQA); and train, based on the training dataset, the GFM to obtain a medically-trained GFM.

Additionally or alternatively, the GFM may include being selected from one of: RadFM, LLaVA-Med, Med-Flamingo, MedDr, and Intern VL.

Additionally or alternatively, the respective images may show the different medical conditions are obtained from OpenI.

Additionally or alternatively, the medical modality may include one of: radiology, pathology, dermatology, ophthalmology, gastroenterology, fundoscopy, chest X-ray, and endoscopy.

According to a 4^{th} aspect, there is disclosed a computing device for processing a sample image of a medical condition, comprising: one or more memories having executable code; and one or more processors coupled to the one or more memories, and configured to execute the code to cause the device to: process, by a set of computer vision models for a medical modality associated with the medical condition, the sample image to obtain a set of determination related to a plurality of predicted diagnoses for the medical condition, wherein the set of computer vision models are selected from a plurality of different sets of computer vision models, wherein respective sets of computer vision models are pretrained with downstream datasets specific to respective medical modalities for different medical conditions; process, by an embedding model, the sample image to determine an associated visual embedding to be used as a query for querying a database, wherein the database include a plurality of entries of value-key pairs associated with respective images of different medical conditions; and wherein a key represents a visual embedding of an image, and an associated value provides textual description of the medical condition shown in said image; query, by at least one computer vision model in the selected set of computer vision models based on vector similarity between the determined visual embedding and the respective keys in the plurality of the entries, the database to retrieve a top-k most similar entries from the database, wherein k is a positive integer; and process, by a medically-trained generalist foundation model (GFM) using the obtained set of determination as reference context and images corresponding with the retrieved top-k most similar entries, the sample image to make a predicted determination on the medical condition shown by the sample image, wherein the medically-trained GFM is trained in accordance with the method of claim 1.

Additionally or alternatively, the vector similarity may be performed based on cosine similarity.

According to a 5^{th} aspect, there is disclosed a non-transitory computer-readable medium comprising executable code, which when executed by a processor of a computing device, cause the device to perform the method of the 1^{st} aspect.

According to 6^{th} aspect, there is disclosed a non-transitory computer-readable medium comprising executable code, which when executed by a processor of a computing device, cause the device to perform the method of any of the 2^{nd} aspect.

Additional benefits and advantages of the disclosed aspects may become apparent from the specification and drawings. The benefits and/or advantages may be individually obtained by the various aspects and features of the specification and drawings, which need not all be provided in order to obtain one or more of such benefits and/or advantages.

### BRIEF DESCRIPTION OF THE DRAWINGS

The accompanying figures, where like reference numerals refer to identical or functionally similar elements throughout the separate views, and which together with the detailed description below are incorporated in and form part of the specification, serve to illustrate various aspects and to explain various principles and advantages in accordance with the present disclosure.

FIG. 1, which includes subplots a-e, provides an overview of the present study, where:
subplot a is a schematic diagram of an exemplary framework for training a generalist foundation model (GFM) to obtain a medically-trained GFM, in accordance with aspects of the present disclosure;
subplot b is a schematic diagram of distribution of training datasets used for training a generalist foundation model (GFM), in accordance with aspects of the present disclosure;
subplot c is a schematic diagram of an exemplary framework vis-à-vis a method for processing a sample image of a medical condition, in accordance with aspects of the present disclosure;
subplot d is a diagram illustrating performance of the trained GFM, in accordance with aspects of the present disclosure; and
subplot e is a diagram illustrating overall performance of the trained GFM on medical image diagnosis tasks, in accordance with aspects of the present disclosure.

FIG. 2 is a flowchart illustrating a method for training a generalist foundation model (GFM), in accordance with aspects of the present disclosure.

FIG. 3 is a flowchart illustrating a method for processing a sample image of a medical condition, in accordance with aspects of the present disclosure.

FIG. 4, which contains subplots a-t, shows experimental results between conventional GFMs and the method of FIG. 3 for processing medical image diagnosis datasets, in accordance with aspects of the present disclosure.

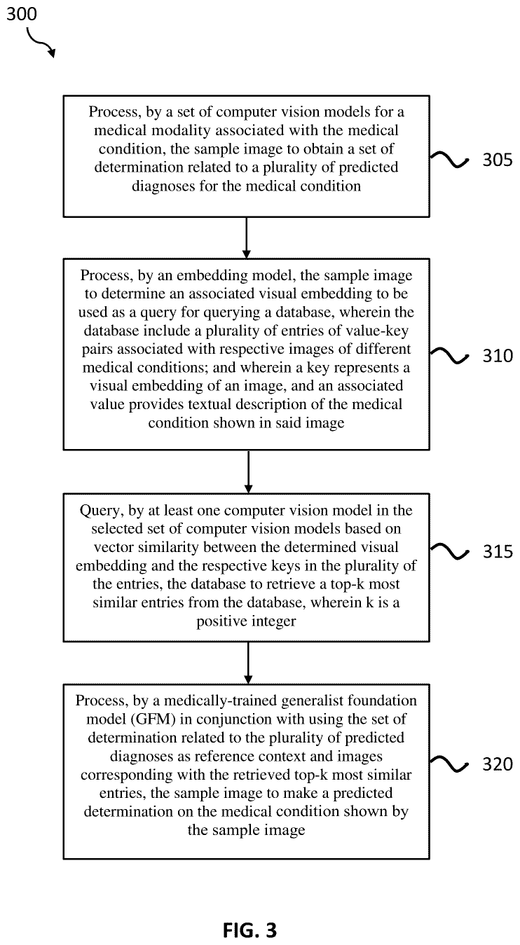

FIG. 5, which includes subplots a-d, shows experimental results of GFMs on medical visual question answering and medical report generation tasks, where:
subplot a shows experimental results between conventional GFMs and the method of FIG. 3 in terms of Closed Question Accuracy and Tokenized F1-Score on VQA-RAD, Slake-VQA, Path-VQA, PMC-VQA, and VQA-Med datasets, in accordance with aspects of the present disclosure;
subplot b shows experimental results between conventional GFMs and the method of FIG. 3 in relation to medical modality and result distribution of the generalist foundation models on the OmnimedVQA dataset, in accordance with aspects of the present disclosure;
subplot c shows experimental results between conventional GFMs and the method of FIG. 3, in terms of ROUGE-L, METEOR, F1-RadGraph on medical report generation datasets MIMIC-CXR and IU-Xray, in accordance with aspects of the present disclosure; and
subplot d shows an example of medical report generation on a chest X-ray image, in accordance with aspects of the present disclosure.

FIG. 6, which includes subplots a-c, provides overall experiment results on medical image diagnosis datasets, where:
subplot a shows experimental results between conventional GFMs and the method of FIG. 3 on medical image diagnosis datasets, in terms of overall ranking of different models or mechanisms on 8 in-domain datasets and the summation of the rankings, in accordance with aspects of the present disclosure;
subplot b shows experimental results between conventional GFMs and the method of FIG. 3 on medical image diagnosis datasets in terms of overall ranking of different models or mechanisms on 12 out-of-domain datasets and the summation of the rankings, in accordance with aspects of the present disclosure; and
subplot c shows a performance comparison between the best specialist (computer vision) model DenseNet-121 and the method of FIG. 3, in accordance with aspects of the present disclosure.

FIG. 7, which includes subplots a-f, show examples of mixture-of-expert diagnosis and retrieval-augmented diagnosis on downstream medical image diagnosis datasets, in accordance with aspects of the present disclosure.

FIG. 8 shows a prompt for diagnosis-guided bootstrapping, whereby {Modality} and {Disease} represent placeholders for the corresponding information, in accordance with aspects of the present disclosure.

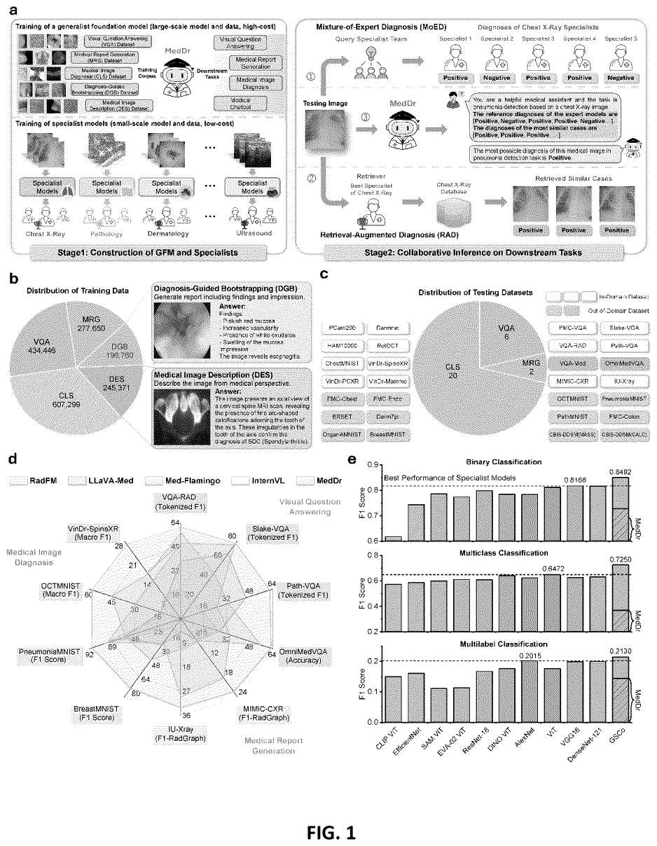

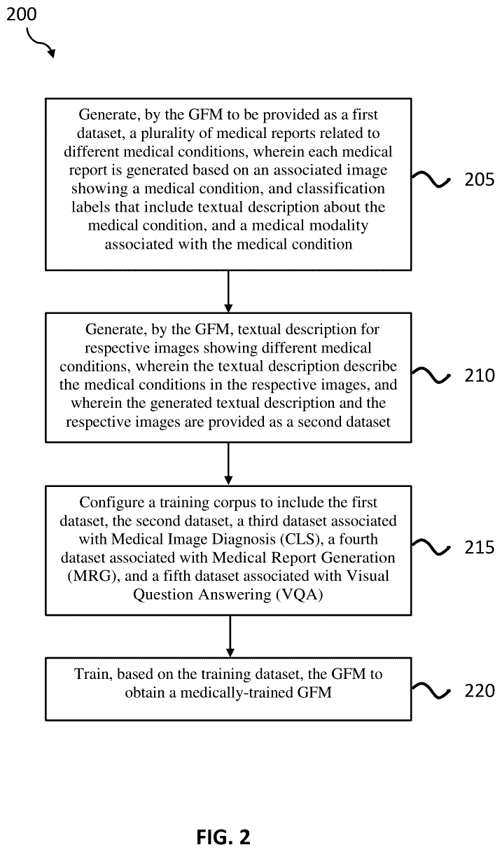

FIG. 9 includes subplots a-c, where:
subplot a shows a situation where a generalist foundation model in the general domain possesses knowledge about diseases but being challenging to correlate the knowledge with querying images, leading to incorrect diagnosis, in accordance with aspects of the present disclosure;
subplot b shows the method of FIG. 2 generates a detailed medical report consisting of findings and impressions, as guided by the correct diagnosis, in accordance with aspects of the present disclosure; and
subplot c shows examples of medical reports generated by the method of FIG. 2 across different medical modalities, in accordance with aspects of the present disclosure.

FIG. 10, which includes subplots a and b, show employment of Mixture-of-Expert Diagnosis by the method of FIG. 3 for processing a sample image of a medical condition, in accordance with aspects of the present disclosure.

FIG. 11, which includes subplots a-c, illustrates retrieval-augmented diagnosis, where:
subplot a shows an embedding model is employed to extract visual embeddings of images in medical image-text pairs, in accordance with aspects of the present disclosure;
subplot b shows each entry in a database comprises meta-information along with its indexed embedding, in accordance with aspects of the present disclosure; and
subplot c shows that during inference, the embedding of the test image is used to query the database and retrieve similar samples, in accordance with aspects of the present disclosure.

FIG. 12 shows examples of Mixture-of-Expert Diagnosis on five downstream medical image diagnosis datasets, in accordance with aspects of the present disclosure.

FIG. 13 shows examples of Retrieval-Augmented Diagnosis on five downstream medical image diagnosis datasets, in accordance with aspects of the present disclosure.

FIG. 14 shows detailed results on binary classification datasets in medical image diagnosis task.

FIG. 15 shows detailed results on multiclass classification datasets in medical image diagnosis task.

FIG. 16 shows detailed results on multilabel classification datasets in medical image diagnosis task.

FIG. 17 shows the performance of generalist foundation models on in-domain visual question answering datasets.

FIG. 18 shows performance of generalist foundation models on the VQA-Med dataset.

FIG. 19 shows performance of generalist foundation models on the OmniMedVQA dataset.

FIG. 20 shows an ablation study of the method of FIG. 3, in accordance with aspects of the present disclosure.

FIG. 21 shows performance of generalist foundation models on the medical report generation task.

FIG. 22 shows performance with retrieval-augmented diagnosis.

FIG. 23 shows detailed information of in-domain medical image diagnosis datasets, in which the dataset name, classification type, dataset size, and label set are listed.

FIG. 24 shows detailed information of out-of-domain medical image diagnosis datasets, in which the dataset name, classification type, dataset size, and label set are listed.

FIG. 25 shows prompt templates for different instruction-tuning dataset vis-à-vis the method of FIG. 2, in accordance with aspects of the present disclosure.

FIG. 26 shows instructions provided for the method of FIG. 3, in accordance with aspects of the present disclosure. Specifically, {Modality} is the placeholder for the name of different medical modalities, Label Set} denotes the candidate label set of the classification tasks, whereas {RAD} and {MoED} are the results of retrieval-augmented diagnosis and mixture-of-expert diagnosis.

FIG. 27 shows specifications of 10 selected computer vision models that encompass example parameters such as the number of parameters, Giga Multiply-Add Operations per Second (GMACs), and the training time per epoch on a dataset, with PneumoniaMNIST, in accordance with aspects of the present disclosure.

FIG. 28 shows hyperparameters used for training each computer vision model, in accordance with aspects of the present disclosure.

FIG. 29 shows the data availability of training datasets, in accordance with aspects of the present disclosure.

FIG. 30 shows the data availability of benchmark datasets, in accordance with aspects of the present disclosure.

FIG. 31 shows the web listing of the public code utilized, in accordance with aspects of the present disclosure.

FIG. 32 is a block diagram of a training manager for training a generalist foundation model (GFM), in accordance with aspects of the present disclosure.

FIG. 33 is a block diagram of a coordination manager for processing a sample image of a medical condition, in accordance with aspects of the present disclosure.

FIG. 34 is a schematic diagram of an exemplary computing device that may be used for performing the method of FIG. 2 or the method of FIG. 3, in accordance with aspects of the present disclosure.

FIG. 35 is a schematic diagram of an exemplary computing device that may be used for performing the method of FIG. 2 or the method of FIG. 3, in accordance with aspects of the present disclosure.

### DETAILED DESCRIPTION

Some portions of the description which follows below are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is generally conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, discussions utilizing terms such as “scanning”, “calculating”, “determining”, “replacing”, “generating”, “initializing”, “outputting”, or the like, refer to the action and processes of a computer system, or similar electronic device, that manipulates and transforms data represented as physical quantities within the computer system into other data similarly represented as physical quantities within the computer system or other information storage, transmission or display devices.

The present specification also discloses apparatus for performing the operations of the methods. Such apparatus may be specially constructed for the required purposes, or may comprise a computer or other device selectively activated or reconfigured by a computer program stored in the computer. The algorithms and displays presented herein are not inherently related to any particular computer or other apparatus. Various machines may be used with programs in accordance with the teachings herein. Alternatively, the construction of more specialized apparatus to perform the required method steps may be appropriate. The structure of a conventional computer will appear from the description below.

In addition, the present specification also implicitly discloses a computer program, in that it would be apparent to the person skilled in the art that the individual steps of the method described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the invention.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a computer. The computer readable medium may also include a hard-wired medium such as exemplified in the Internet system, or wireless medium such as exemplified in the GSM, GPRS, 3G, 4G, 5G, NR mobile communication systems, as well as other wireless communication systems/standards such as Bluetooth, ZigBee, or Wi-Fi. The computer program when loaded and executed on such a computer effectively results in an apparatus that implements aspect(s) of the present disclosure.

Aspect(s) of the present disclosure may be implemented as hardware modules. More particularly, in the hardware sense, a module is a functional hardware unit designed for use with other components or modules. For example, a module may be implemented using discrete electronic components, or it can form a portion of an entire electronic circuit such as an Application Specific Integrated Circuit (ASIC) or Field Programmable Gate Array (FPGA). Numerous other possibilities exist, as known in the art. Those skilled in the art may also appreciate that the system is implementable as a combination of hardware and software modules.

Aspects of the present disclosure provide methods and corresponding devices for training a generalist foundation model (GFM), and also for processing sample images of (different) medical conditions, the latter of which is performed based at least in part on the trained GFM. For avoidance of doubt, a medical condition generally may refer to a medical disease, any specific health issue or illness that can be diagnosed by a healthcare provider based on symptoms, medication use, or diagnostic testing. Discussions that follow below set out the disclosed subject matter, in accordance with various aspects of the present disclosure.

For completeness, it is clarified that reference made to the definition of format: “[ref. X]” in any paragraph(s) in the description is to be construed to refer to the corresponding citation “X” in the “References” section of the present specification. For example, [ref. 10] refers to citation [10] listed at the “References” section of the present specification, while [ref. 21-24] correspondingly refers to citations [21]-[24] mutatis mutandis.

Specifically, the method for processing a sample image is based on an exemplary cooperative framework termed as Generalist-Specialist Collaboration (hereinafter “GSCo”), to explore the synergy between a GFM and specialist (computer vision) models, according to aspects of the present disclosure. FIG. 1, subplot a, is a schematic diagram of the framework for training a GFM to obtain a medically-trained GFM, in accordance with aspects of the present disclosure. The GFM may be based on one known in the art [e.g. see ref. 9], but is not to be construed to be limiting as such. The GSCo framework may comprise two stages: the construction (i.e. training) of GFM and specialist models, and subsequent collaborative inference on downstream tasks.

At the construction stage, the medically-trained GFM (hereinafter termed “MedDr”) may be developed based on a large-scale training corpus of medical image-text pairs across various modalities. Meanwhile, a series of lightweight specialist models may be selected and tailored to specific downstream tasks with much lower computational consumption. It is to be appreciated that downstream tasks may include medical image analysis tasks, such as e.g. pneumonia detection, lesion classification or the like.

At the collaborative inference stage, two mechanisms, namely Mixture-of-Expert Diagnosis (hereinafter termed “MoED”) and Retrieval-Augmented Diagnosis (hereinafter termed “RAD”) are herein disclosed to enable synergistic cooperation between MedDr and the specialist models, in accordance with aspects of the present disclosure.

In particular at the construction stage, the development of an advanced medical GFM is of focus. To curate a large-scale multi-modal training corpus, two datasets are hereby introduced: the first dataset is known as the Diagnosis-Guided Bootstrapping (DGB) dataset and the second dataset is known as the Medical Image Description (DES) dataset. The DGB dataset may be constructed based on abundant medical image diagnosis data [ref. 30-35], which aims to enhance the intrinsic disease diagnosis capabilities of the GFM. In contrast to conventional methods [ref. 12, 14] that rely solely on textual information of image-text pairs to generate instruction-tuning data, the DGB dataset is generated by integrating both visual and textual information. The GFM may be utilized in the general domain to generate detailed medical reports, including findings and conclusions, based on diagnosis information (e.g., classification labels). Guided by the human-verified annotations, the generated data not only demonstrates increased reliability but also significantly enriches the depth and diversity of visual information conveyed in the text.

Additionally, the DES dataset may be utilized to broaden the scope of the training data, by incorporating image-based case studies corresponding to diverse medical conditions from OpenI [ref. 36]. The GFM [ref. 9] may also be utilized to rewrite the text description of the case studies, and to remove any information that may be derived from the corresponding image. In addition to the DGB and DES datasets, the Medical Image Diagnosis (CLS) [ref. 31], Medical Report Generation (MRG) [ref. 21], and Visual Question Answering (VQA) [ref. 37] datasets may be incorporated too for training the GFM. Overall, the training corpus may consist of more than 2 million samples across five distinct types of training datasets, covering a wide range of medical modalities. FIG. 1, subplot b, shows the distribution of the training corpus and two samples from the DGB and DES datasets respectively, in accordance with aspects of the present disclosure.

Building upon the training corpus, MedDr may then be developed, which is considered the largest open-source generalist foundation model for medicine consisting of 40B parameters. Compared to other conventional generalist foundation models for medicine [ref. 12-14], MedDr advantageously exhibits superior capabilities in medical image analysis across more diverse medical modalities, including radiology, pathology, dermatology, ophthalmology, and gastroenterology.

Moreover, MedDr demonstrates advanced in-context learning and instruction-following capability, and fosters collaboration between the trained GFM and the specialist models.

In the collaborative inference stage, the synergistic relationship between MedDr and specialist models is disclosed below. To exploit the generalist's in-context learning abilities alongside the specialists' domain expertise, Mixture-of-Expert Diagnosis (MoED), and Retrieval-Augmented Diagnosis (RAD) are disclosed as mechanisms for collaboration between the trained GFM and the specialist models. MoED diagnosis aims to supplement and boost the GFM with prediction results from the specialist models. The paradigm of MoED [ref. 38], which ensembles insights from multiple experts, has previously been explored in the art to enhance the robustness of the predictions [ref. 39-41]. In MoED, the outputs of the specialist models act as the reference context and they are then provided to MedDr with the testing image together. Thereafter, MedDr is required to provide a predicted diagnosis, by considering both the content of the testing image and the results provided by the specialist models. Different from MoED, which exploits the inherent expert knowledge of the specialist models, retrieval-augmented diagnosis is further proposed to fully leverage the broad medical knowledge embedded within the existing data).

In contrast to previous medical generalist foundation models [ref. 12-15] that relied solely on the internal knowledge of the models to make diagnoses, Retrieval-Augmented Generation (RAG) [ref. 42] is incorporated under the proposed GSCo framework to leverage external knowledge, thereby enhancing model accuracy and reliability. In the proposed RAD mechanism, each specialist model may serve as a retriever, using the visual embedding of the testing image as the query to retrieve the most similar “K” samples in the database, where K is a positive integer. In various examples, the best specialist model (from all selected specialist models employed for RAG) may function to perform the role of the retriever. The information from the retrieved samples is then provided to MedDr as a contextual reference to assist medical image analysis. So, MoED and RAD may collectively provide helpful guidance to MedDr, based on the expertise of the specialists. Meanwhile, for its role as a decision-maker, MedDr integrates its intrinsic knowledge with external knowledge to render the final diagnosis.

To comprehensively evaluate MedDr and the proposed GSCo framework, a large-scale benchmark is curated. Compared with conventional works [ref. 12-15, 43], the curated benchmark excels in both diversity and magnitude. As shown in FIG. 1, subplot c, the benchmark encompasses diverse medical datasets such as medical image diagnosis, visual question answering, medical report generation tasks, etc. Specifically, there are 14 in-domain datasets and 14 out-of-domain datasets, resulting in a total of 250,000 samples. To assess the improvements brought by GSCo to GFM on medical image diagnosis datasets, where specialists may yield superior results due to their domain-specific knowledge, 20 distinct medical image diagnosis datasets, including 11 medical modalities, are integrated. Experiments are conducted on conventional GFMs for comparison to demonstrate the superiority of MedDr.

FIG. 1, subplot d, shows comparison among GFMs on 10 benchmark datasets that span different medical tasks and modalities. It may be seen that MedDr consistently surpasses other conventional GFMs from both medical and general domains by a large margin. Experiments are also performed to validate the effectiveness of GSCo. FIG. 1, subplot e, illustrates the performance of MedDr on medical image diagnosis tasks. Despite the competitive performance of specialist models due to their domain-specific knowledge, the proposed GSCo framework further improves the performance of MedDr and surpasses all specialist models. These experimental results highlight the significance of the proposed GSCo framework, representing a paradigm shift in the clinical application of GFMs. This transition moves away from utilizing separate models for medical tasks independently to fostering collaboration between GFMs and specialist models.

The advantages of the GSCo framework are twofold: Firstly, GSCo is effective. Compared with the independent use of either GFMs or specialist models, GSCo demonstrates superior performance, particularly on out-of-domain datasets, showcasing its advanced generalizability. Secondly, GSCo is efficient. When confronted with out-of-domain tasks or data, rather than investing substantial resources to fine-tune the GFM, it may efficiently adapt lightweight specialist models with minimal consumption, indicating its scalability and sustainability.

The following set out the contributions of the proposed framework, in accordance with aspects of the present disclosure.

Generalist-Specialist Collaboration (GSCo) is introduced, in the authors' knowledge as being the first collaborative framework to explore the synergy between the GFM and specialist models. GSCo harvests the generalist's in-context learning ability and the specialists' domain expertise, to enable precise medical image analysis on diverse medical tasks. This synergistic paradigm not only broadens the functionalities of the GFM with efficient resource utilization but also ensures scalability and sustainability, thereby catalyzing the advancement of generalizable AI in the medical field.

MedDr, being the largest open-source generalist foundation model tailored for medicine, is also introduced. In the development of MedDr, diagnosis-guided bootstrapping (DGB) and medical image description (DES) are introduced to enhance the diversity of the training corpus. As a result, MedDr is capable of handling various medical modalities and tasks, achieving state-of-the-art performance in downstream tasks and outperforming other conventional GFMs. It is to be appreciated that downstream tasks may include medical image analysis tasks, such as e.g. pneumonia detection, lesion classification or the like. Additionally, MedDr may excel in instruction-following and in-context learning, providing a better foundation for collaboration with specialist models.

Two cooperative mechanisms, mixture-of-expert diagnosis (MoED) and retrieval-augmented diagnosis (RAD) are also disclosed in order to facilitate collaboration between MedDr and the specialist models. MoED incorporates the diagnoses of specialists as guidance, while RAD utilizes the specialists to retrieve the most similar cases for reference. The results of MoED and RAD are combined together as the context information to be provided to MedDr as guidance.

A large-scale benchmark is also established, which comprises 28 datasets with 250,000 test samples, covering more than ten medical modalities across various medical tasks. Extensive experiments are conducted on the benchmark, which demonstrate the superior capabilities of MedDr and validate the efficacy of the proposed GSCo framework.

FIG. 2 is a flowchart illustrating a method 200 for training a generalist foundation model (GFM), in accordance with aspects of the present disclosure. As an example, the GFM used may be based on one known in the art [e.g. ref. 9]. This method 200 corresponds to the construction (i.e. training) stage for the GFM and specialist models, under the GSCo framework. The method 200 may be realized as a computer-implemented method. The operations of method 200 may be implemented at, and performed by a computer device 3400, 3500, as shown in FIGS. 34-35, or its components. In some examples, a computer device 3400, 3500 may execute a set of instructions to control the functional elements of the computer device 3400, 3500 to perform the functions described below. Additionally or alternatively, a computer device 3400, 3500 may perform aspects of the functions described below using special-purpose hardware.

At 205, the method 200 may comprise generating, by the GFM to be provided as a first dataset, a plurality of medical reports related to different medical conditions, wherein each medical report is generated based on an associated image showing a medical condition, and classification labels that include textual description about the medical condition, and a medical modality associated with the medical condition.

At 210, the method 200 may comprise generating, by the GFM, textual description for respective images showing different medical conditions, wherein the textual description describe the medical conditions, based on diagnostically relevant visual features shown in the respective images, and wherein the generated textual description and the respective images are provided as a second dataset.

At 215, the method 200 may comprise configuring a training dataset to include the first dataset, the second dataset, a third dataset associated with Medical Image Diagnosis (CLS), a fourth dataset associated with Medical Report Generation (MRG), and a fifth dataset associated with Visual Question Answering (VQA).

At 220, the method 200 may comprise training, based on the training dataset, the GFM to obtain a medically-trained GFM.

FIG. 3 is a flowchart illustrating a method 300 for processing a sample image of a medical condition, in accordance with aspects of the present disclosure. This method 300 corresponds to the inference stage, under the GSCo framework. The method 300 may be realized as a computer-implemented method. The operations of method 300 may be implemented at, and performed by a computer device 3400, 3500, as shown in FIGS. 34-35, or its components. In some examples, a computer device 3400, 3500 may execute a set of instructions to control the functional elements of the computer device 3400, 3500 to perform the functions described below. Additionally or alternatively, a computer device 3400, 3500 may perform aspects of the functions described below using special-purpose hardware.

At 305, the method 300 may comprise processing, by a set of computer vision models for a medical modality associated with the medical condition, the sample image to obtain a set of determination related to a plurality of predicted diagnoses for the medical condition, wherein the set of computer vision models are selected from a plurality of different sets of computer vision models, wherein respective sets of computer vison models are pretrained with downstream datasets specific to respective medical modalities for different medical condition. To clarify, the plurality of different sets of computer vision models refer to the different diverse specialist models, under the GSCo framework.

In certain examples, the method 300 may alternatively first select the set of computer vision models from the plurality of different sets of computer vison models, prior to performance of step 305.

At 310, the method 300 may comprise processing, by an embedding model, the sample image to determine an associated visual embedding to be used as a query for querying a database, wherein the database include a plurality of entries of value-key pairs associated with respective images of different medical conditions; and wherein a key represents a visual embedding of an image, and an associated value provides textual description of the medical condition shown in said image.

At 315, the method 300 may comprise querying, by at least one computer vision model in the selected set of computer vision models based on vector similarity between the determined visual embedding and the respective keys in the plurality of the entries, the database to retrieve a top-k most similar entries from the database, wherein k is a positive integer.

At 320, method 300 may comprise processing, by a medically-trained generalist foundation model (GFM) in conjunction with using the set of determination related to the plurality of predicted diagnoses as reference context and images corresponding with the retrieved top-k most similar entries, the sample image to make a predicted determination on the medical condition shown by the sample image, wherein the medically-trained GFM is trained in accordance with the method 200 discussed vis-à-vis FIG. 2. To clarify, the medically-trained GFM refers to the proposed MedDr.

### Results

### Comprehensive Evaluation Benchmark in Medicine

In this study, to thoroughly assess the model's performance of medical tasks, a large-scale benchmark, encompassing 28 public datasets comprising about 250,000 samples, is curated. Compared with previous works [ref. 12-15, 43], this benchmark excels in both diversity and magnitude. The datasets within the benchmark are carefully selected to include clinically pertinent tasks such as medical image diagnosis, visual question answering, and medical report generation. Additionally, the benchmark is configured to span a diverse range of medical image modalities, including radiology, pathology, dermatology, ophthalmology, gastroenterology, etc., ensuring a comprehensive evaluation of the model's capabilities across various medical conditions.

### Medical Image Diagnosis

Medical Image Diagnosis is one of the most fundamental tasks in medicine, which requires the model to diagnose the queried images within a predefined label set. In the disclosed benchmark, 20 distinct medical image diagnosis datasets, encompassing approximately 100,000 testing samples and 11 medical modalities, are integrated. The benchmark has two prominent features. First, the benchmark covers diverse medical conditions. For instance, VinDr-SpineXR [ref. 25] and FMC-Chest [ref. 27] are challenging radiology datasets that focus on spine and chest X-ray images, respectively. HAM10000 [ref. 31] and DermNet [ref. 44] are both datasets related to skin diseases, with the former focusing more on dermatoscopic images and the latter more on clinical images. RetOCT [ref. 45] and BRSET [ref. 46] focus on ocular diseases, albeit with different medical modalities. Second, the benchmark incorporates different classification types. For example, PneumoniaMNIST [ref. 26] and BreastMNIST [ref. 26] are binary classification datasets. While providing an available answer (i.e., Positive or Negative) for a binary classification task is straightforward for a GFM, achieving accurate predictions is indeed challenging. FMC-Endo [ref. 27] and OCTMNIST [ref. 26] are multi-class classification datasets with label sets consisting of 5 and 4 labels, respectively.

For each given image, the model is required to predict only one label among the predefined label sets. The most challenging task is multi-label classification, where each test image may possess multiple, or even no, labels. For example, ChestMNIST [ref. 33] is a multi-label classification dataset consisting of 15 categories, with each sample potentially having one or more positive labels. Such tasks impose greater demands on the model's disease diagnosis and task understanding capabilities than binary and multi-class classification tasks. To facilitate comparative analysis, these medical image diagnosis datasets are categorized into two groups: in-domain datasets and out-of-domain datasets, based on whether the training split of the dataset is included in the training corpus of MedDr. The data tables in FIGS. 23-24 provide further detailed information about these datasets. For evaluation metrics, accuracy and macro-F1 are employed for these medical image diagnosis tasks. For binary classification datasets, the results are presented in terms of accuracy. For multi-class and multi-label classification datasets, due to the unbalanced distribution of samples across different classes, the results are reported in terms of the Macro-F1 score. Further details regarding the metric employed are discussed in sections below.

### Visual Question Answering

Visual Question Answering (VQA) task requires the model to answer the question based on the given image, requiring a thorough comprehension of both the visual and textual information. Experiments are conducted on 6 distinct VQA datasets, comprising 4 in-domain datasets: VQA-RAD [ref. 37], Slake-VQA [ref. 47], Path-VQA [ref. 19], and PMC-VQA [ref. 48], as well as 2 out-of-domain datasets: VQA-Med [ref. 20] and OmniMedVQA [ref. 28]. The diversity inherent in these benchmark datasets allows extraction of valuable insights across various domains and facilitates a thorough evaluation of model performance. For example, VQA-RAD, Slake-VQA, and VQA-Med datasets primarily focus on radiology data, such as CT, MRI, and X-ray images, while Path-VQA is mainly about pathology data. In contrast, the PMC-VQA and OmniMedVQA datasets are larger in scale, and cover a broader spectrum of medical modalities. PMC-VQA is built on PubMed while OmniMedVQA is derived from a wide range of medical datasets. Notably, aside from OmniMedVQA, which consists of multiple-choice questions, the other datasets contain free-form questions. For the evaluation of OmniMedVQA, accuracy is employed as a metric. For other datasets, following MultiMedEval [ref. 49], the results are evaluated using both natural language generation (NLG) and classification metrics.

### Medical Report Generation

Medical Report Generation (MRG) involves the model's ability to enumerate all observations and deliver a diagnosis based on the analysis of the medical image. This task presents a significant challenge, as the model is required to accurately capture the complexities inherent in the medical images being evaluated. To assess the performance of MRG, experiments are conducted on two benchmark datasets, i.e., MIMIC-CXR [ref. 21] and IU-Xray [ref. 22]. Both datasets focus on chest X-ray images and offer detailed medical reports that summarize patient conditions. For evaluation of the models, both NLG metrics and model-based metrics in MultiMedEval [ref. 49] are utilized. Further details regarding the metric employed in VQA and MRG tasks are set out in the sections below.

### MedDr Demonstrates Superior Performance in Medical Image Diagnosis

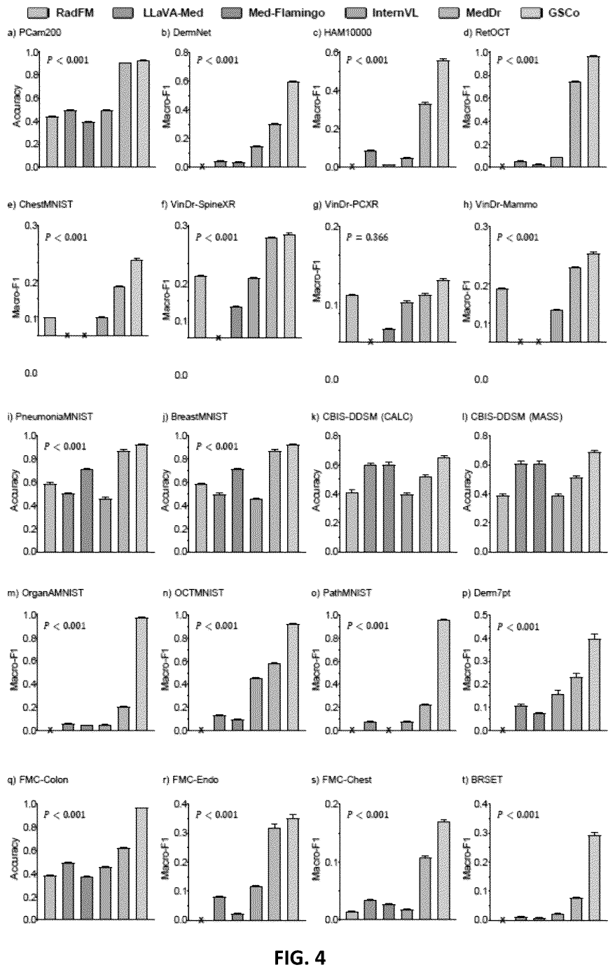

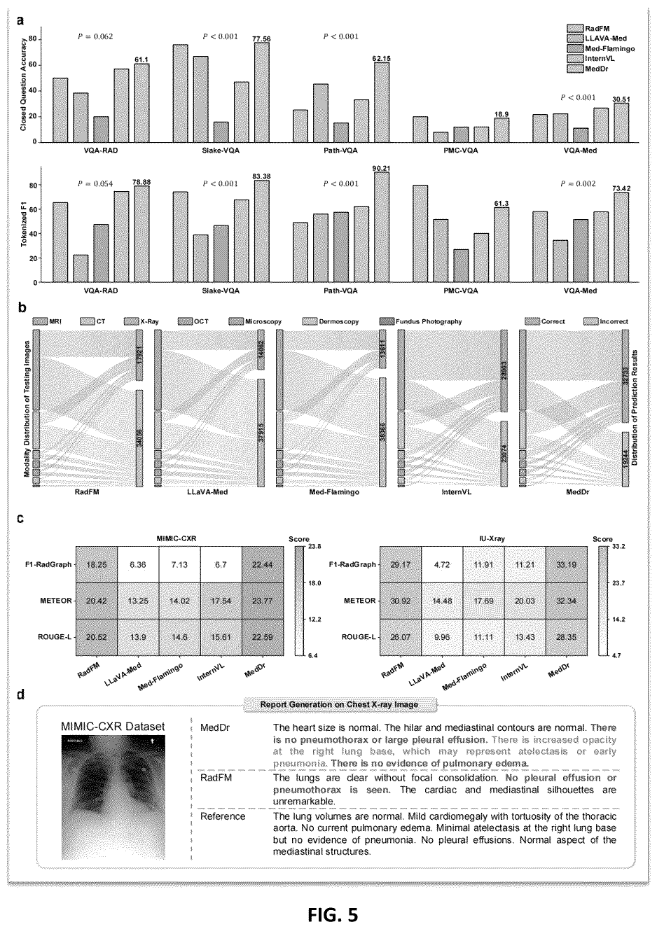

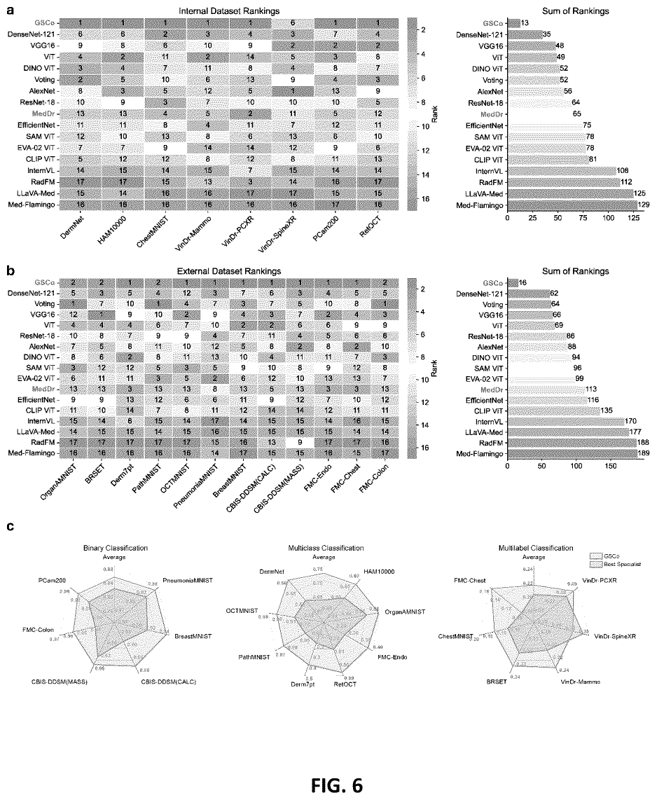

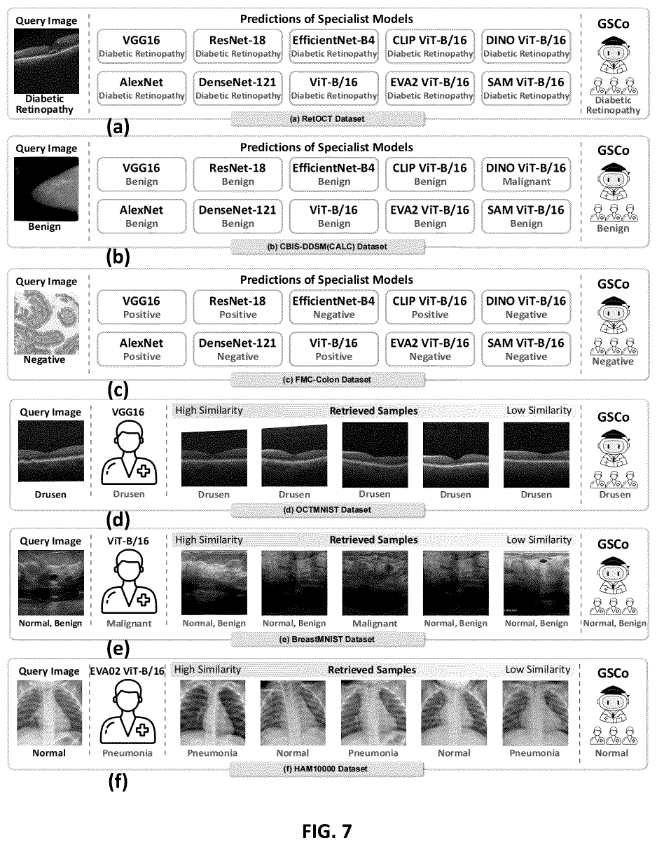

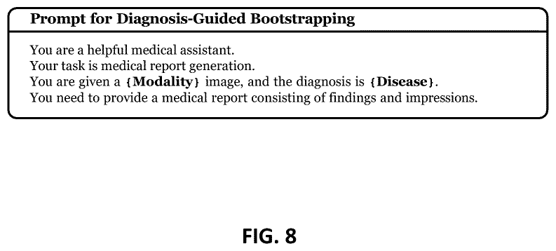

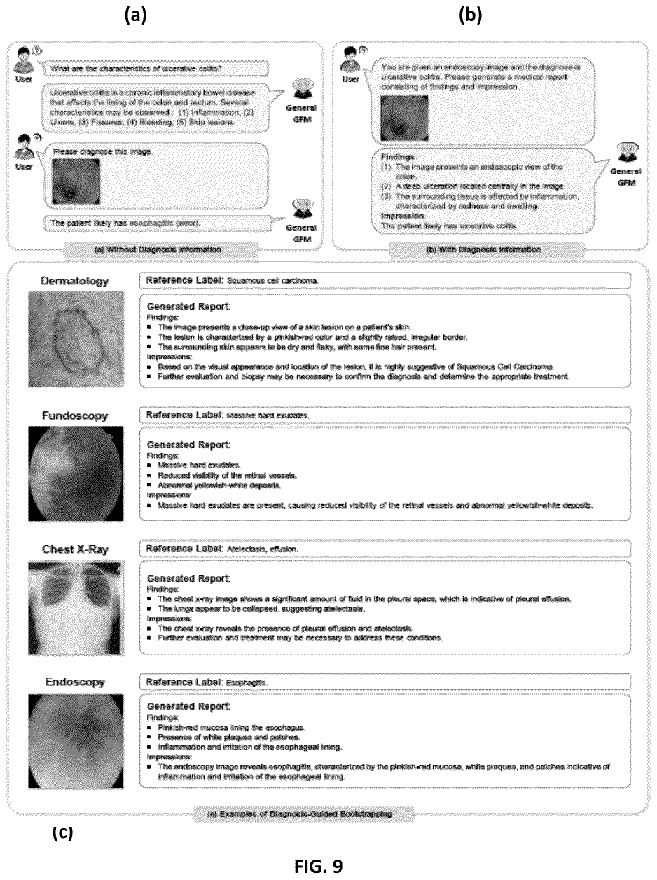

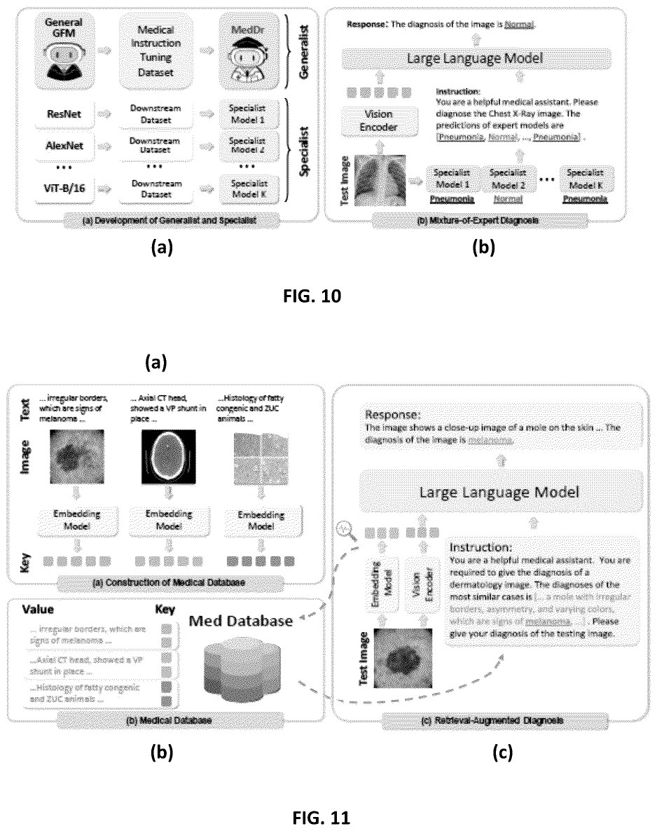

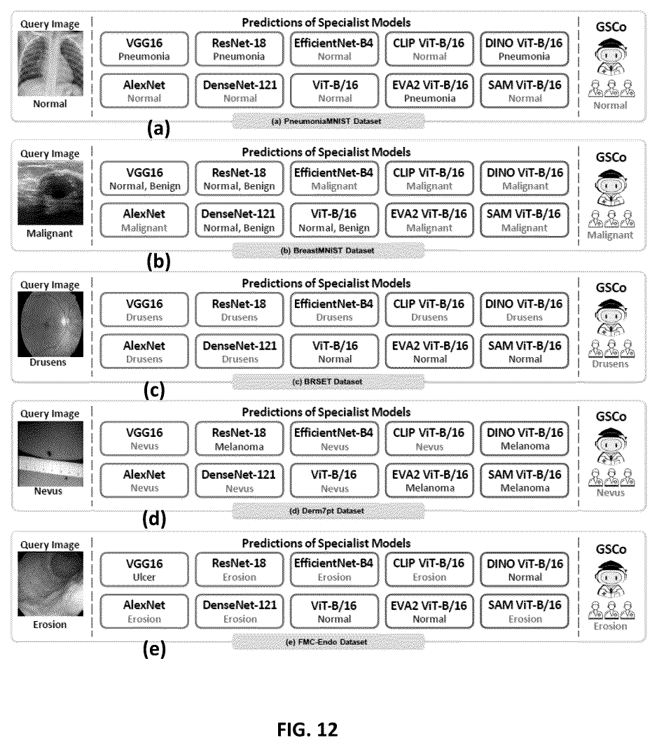

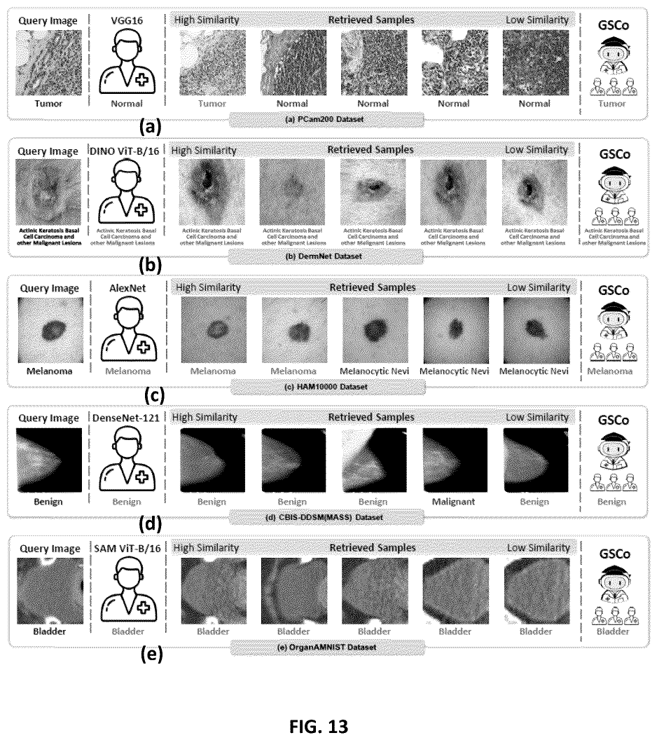

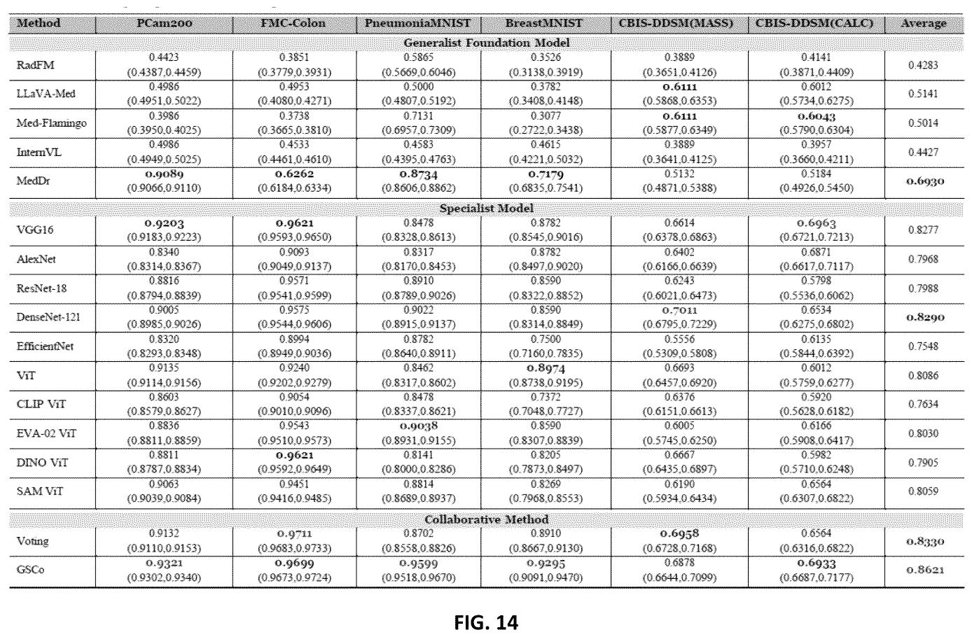

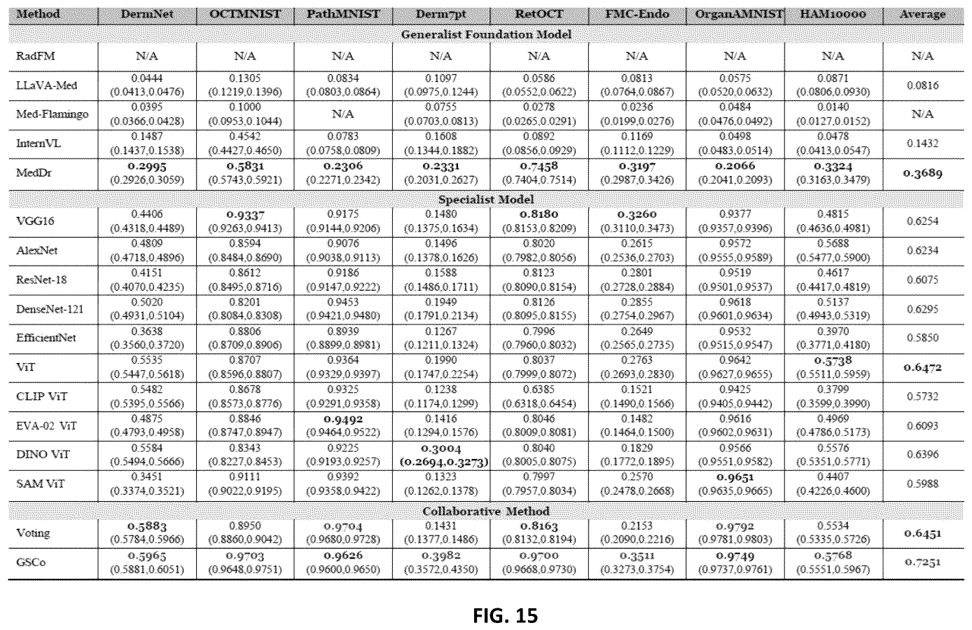

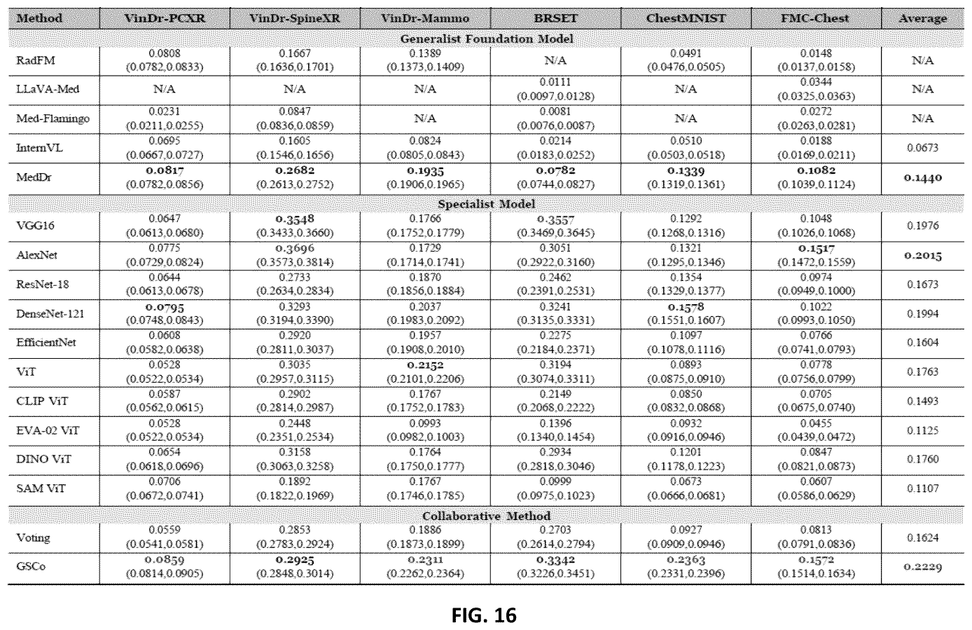

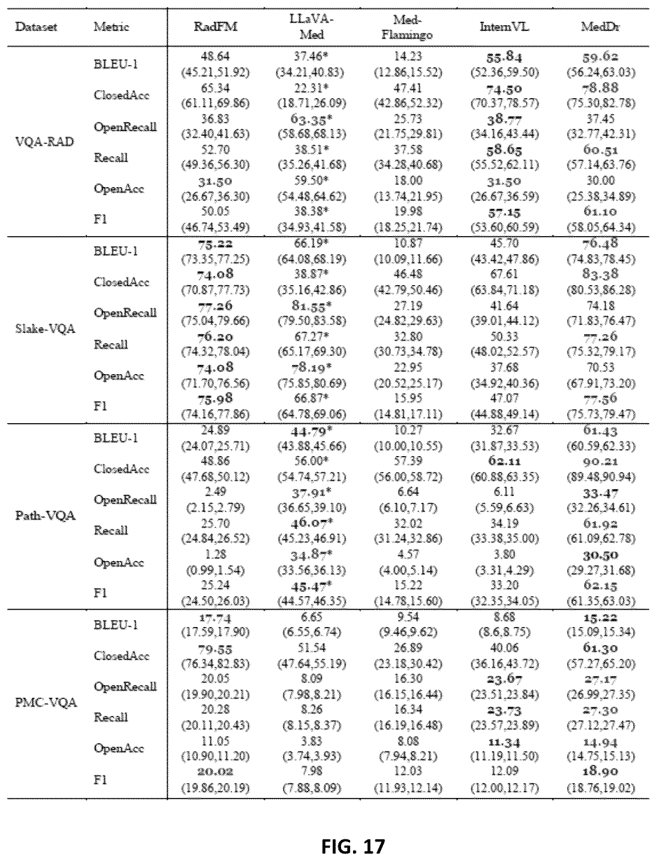

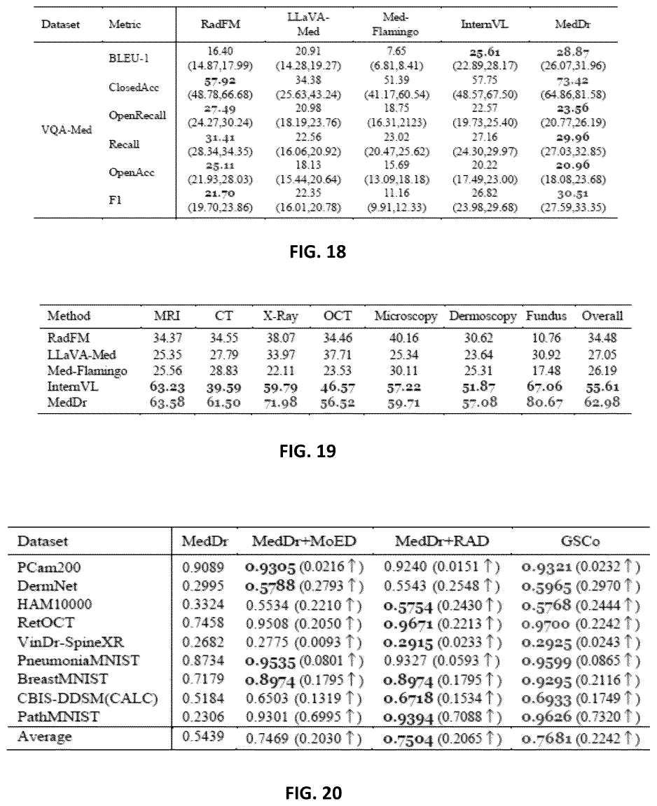

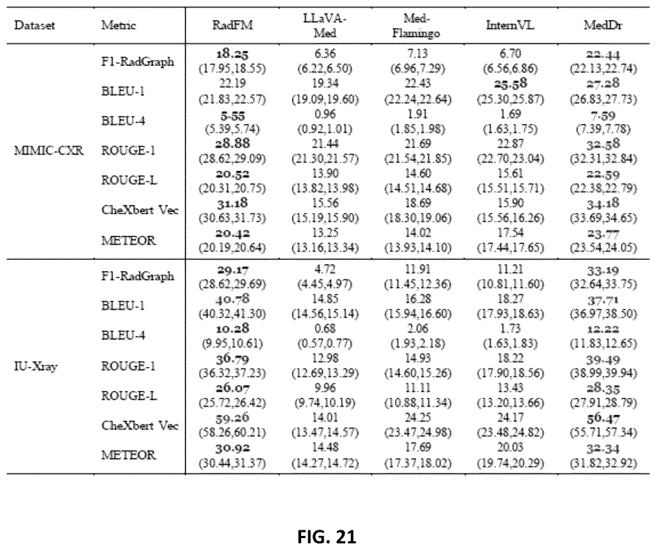

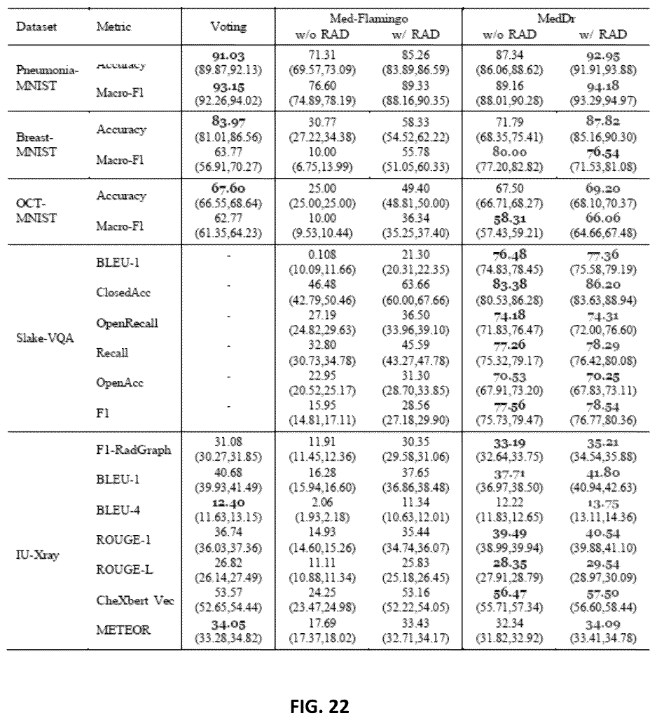

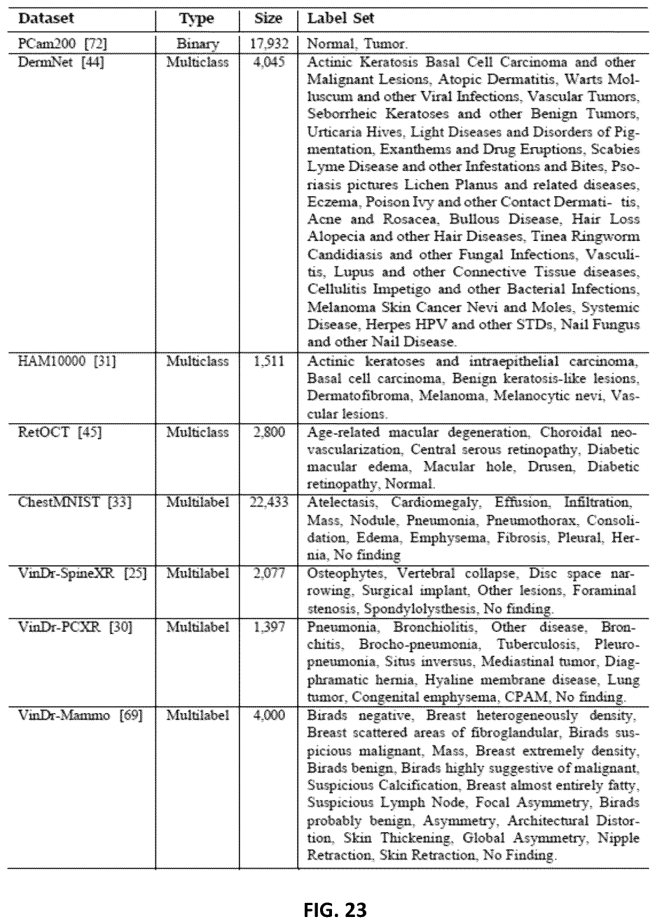

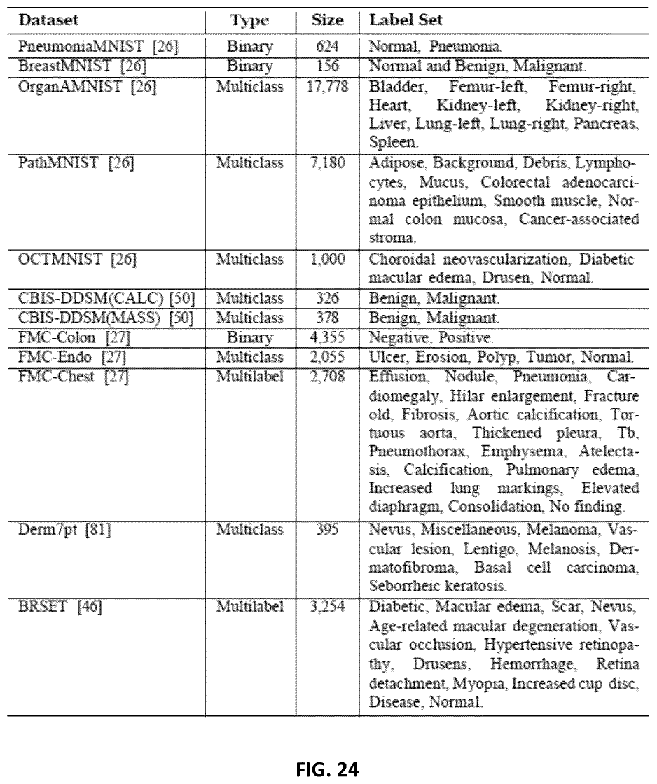

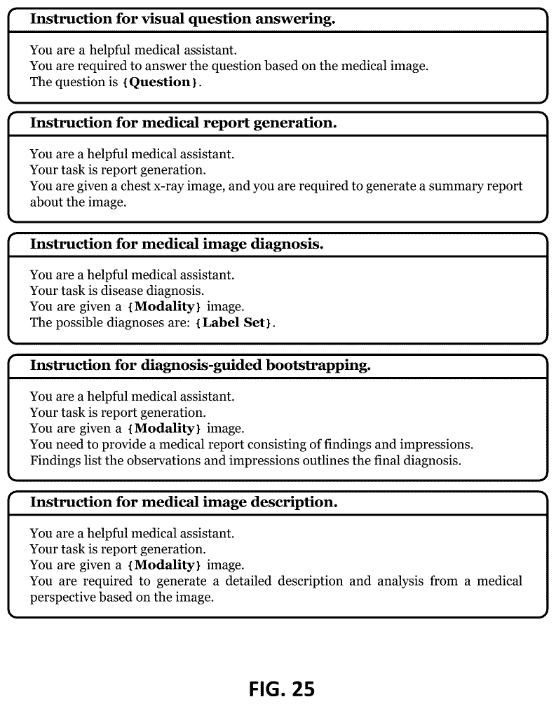

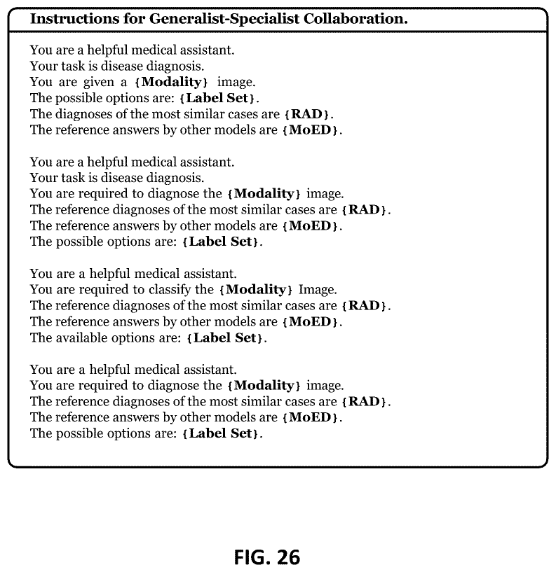

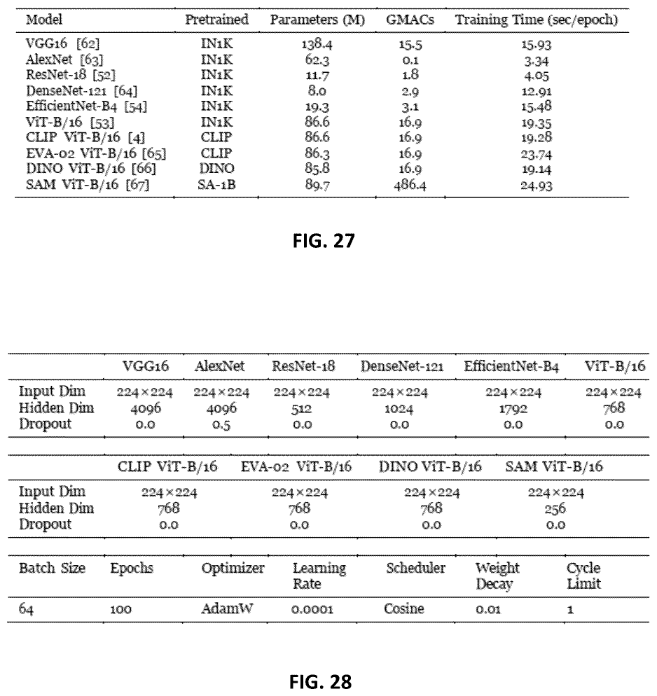

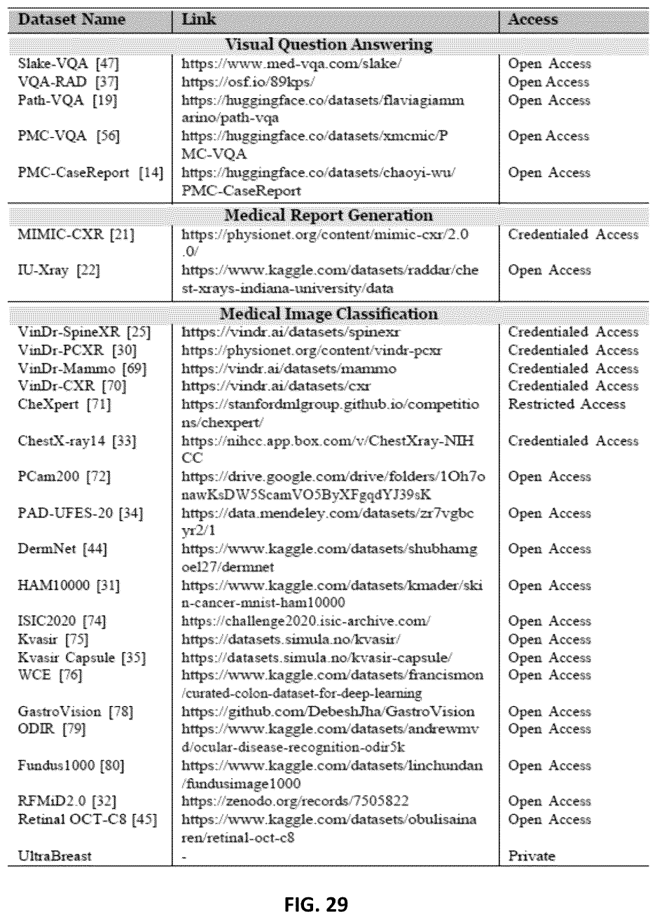

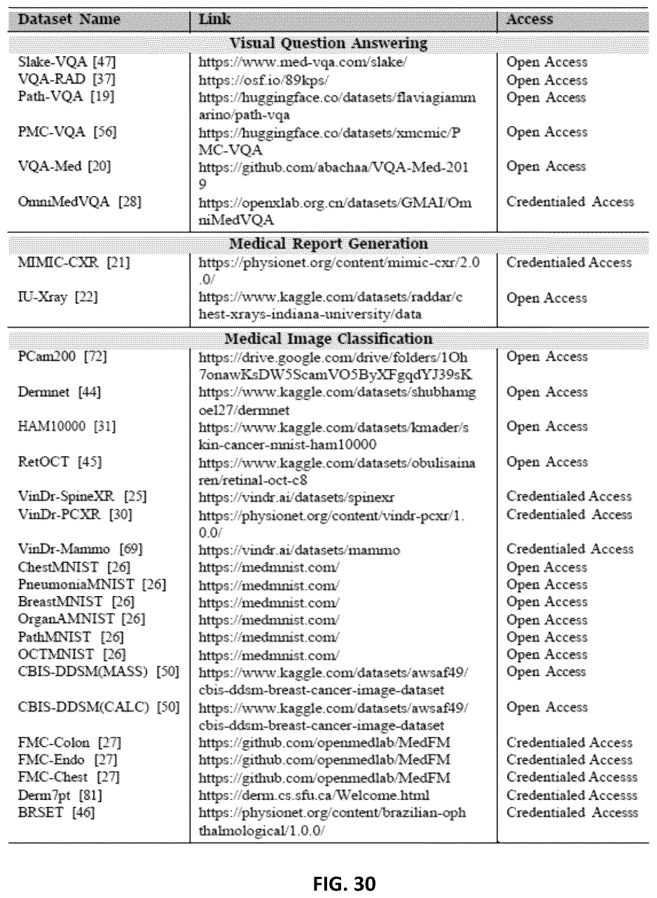

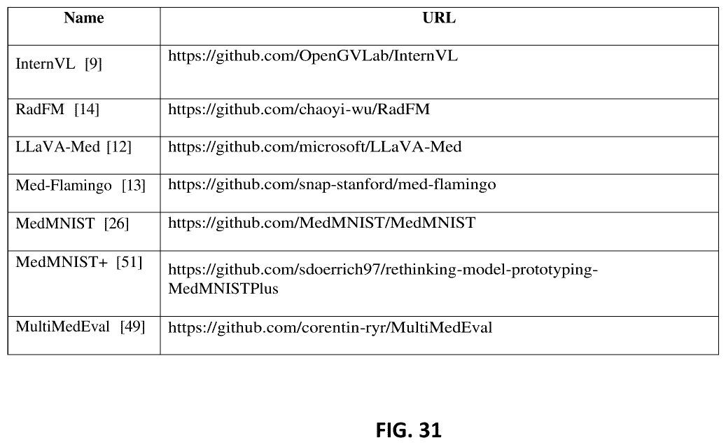

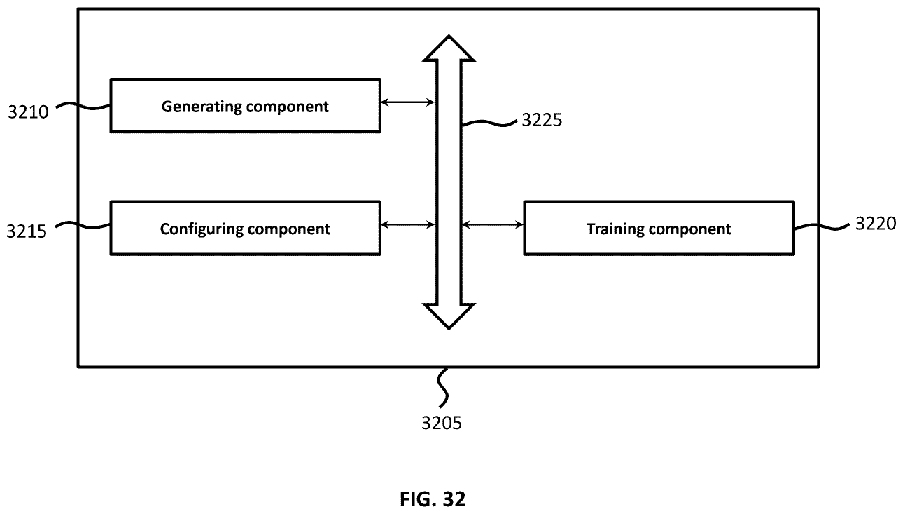

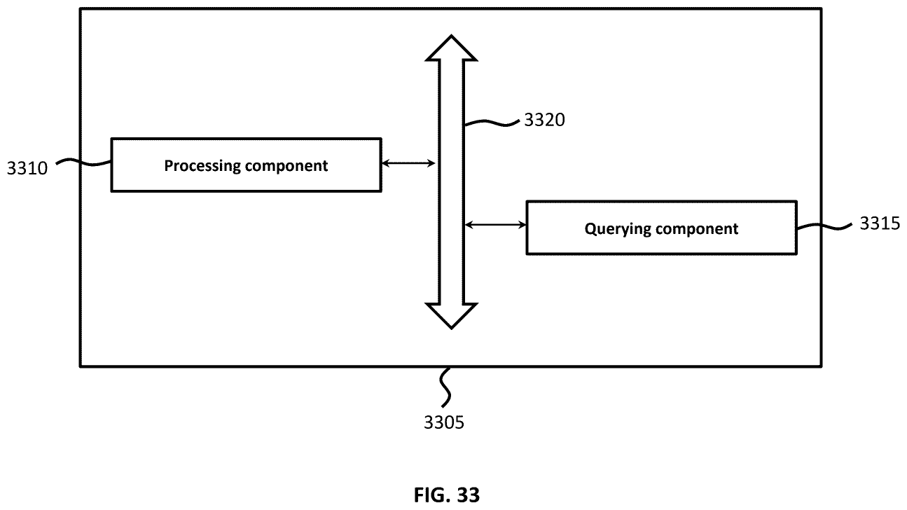

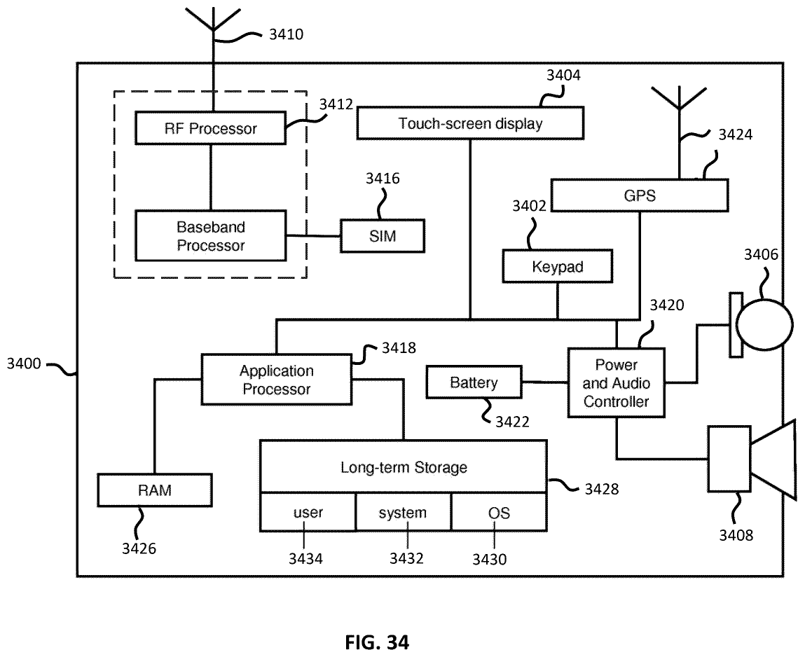

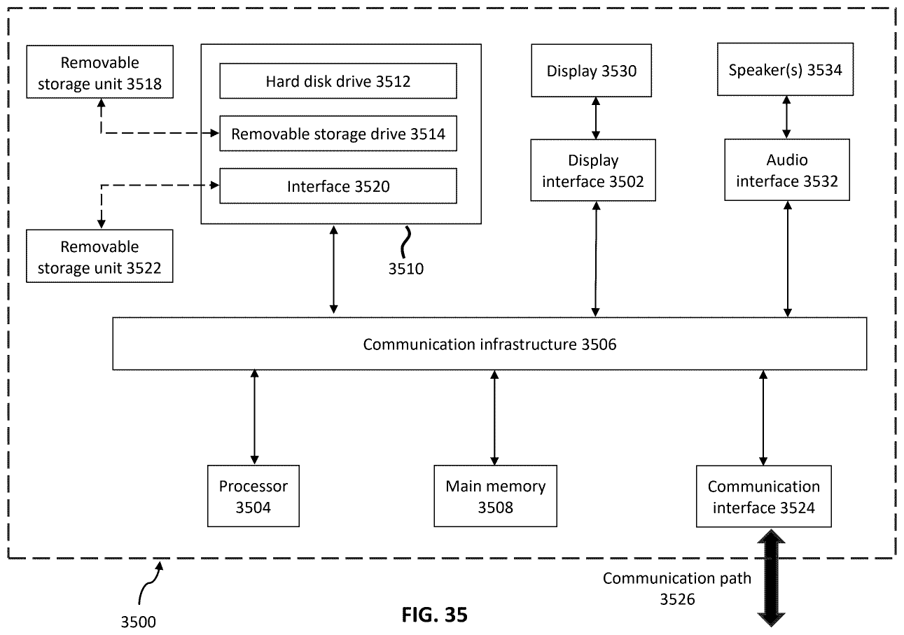

The performance of the GFMs on medical image diagnosis tasks is first explored. It should be noted that some GFMs are unable to produce appropriate responses used for evaluation on specific datasets, so they are not included in the comparison. If the performance of MedDr is the best one compared to other GFMs, the P-value would be presented. Accordingly, the experimental results are depicted in FIG. 4, which contains subplots a-t. Specifically, subplots a-h of FIG. 4 depict the in-domain results, whereas subplots i-t of FIG. 4 depict the out-of-domain results. For binary classification datasets (e.g., PCam200), the results are reported in terms of accuracy. For multi-class (e.g., DermNet) and multi-label datasets (e.g., ChestMNIST), the results are presented in terms of macro-F1 score.

Overall, MedDr outperforms other GFMs significantly and demonstrates its superiority in three aspects. Firstly, MedDr is proficiency in instruction following. It is noted that RadFM, LLaVA-Med, and Med-Flamingo occasionally may struggle to generate appropriate outputs for tasks that involve multiple labels. For instance, in multi-class classification tasks where only one label exists, RadFM, LLaVA-Med, and Med-Flamingo may either output the entire label set or merely rephrase the instructions, suggesting their inferior instruction following capabilities. In contrast, MedDr effectively adheres to instructions across a diverse range of tasks, consistently generating coherent and contextually appropriate outputs.

Secondly, MedDr is able to handle a broader scope of medical modalities. RadFM [ref. 14] is a GFM focusing on the radiology data, so that it achieves commendable performance on radiology datasets such as VinDr-SpineXR [ref. 25] and VinDr-PCXR [ref. 30]. However, performance of RadFM diminishes, when applied to other medical modalities, indicating its limited generalization capabilities. In comparison, MedDr is capable of processing diverse medical modalities, including radiology, pathology, dermatology, ophthalmology, gastroenterology, etc.

Thirdly, MedDr excels in medical image diagnosis. LLaVA-Med [ref. 12] and Med-Flamingo [ref. 13] are mainly trained on visual question answering datasets. Overall performance of LLaVA-Med, and Med-Flamingo in medical image diagnosis tasks is still far from satisfactory. Meanwhile, InternVL [ref. 9] may follow the instructions well, but its performance still lags MedDr by a large margin due to its limited inherent medical knowledge. For instance, on the FMC-Endo [ref. 27] dataset, MedDr obtains a 32.0% Macro-F1 score and significantly outperforms InternVL (11.7% Macro-F1 score, P<0.001, subplot r of FIG. 4).

Notably, in some binary classification datasets, such as CBIS-DDSM (CALC) [ref. 50] and CBIS-DDSM (MASS) [ref. 50], LLaVA-Med, Med-Flamingo and Intern VL struggle to distinguish between the two classes, consistently assigning the same label to all samples. Although these models may achieve higher accuracy, their F1 score is 0. This lack of differentiation indicates these models may fail to capture the pertinent features necessary for effective classification, thereby undermining the reliability of their results. In contrast, MedDr achieves outstanding performance across various medical modalities and classification tasks, thereby highlighting its advanced capabilities in medical image analysis.

### MedDr Excels in Visual Question Answering and Medical Report Generation

In this section, the evaluation of GFMs in visual question answering and medical report generation tasks is discussed. The results of VQA tasks are depicted in subplots a-b of FIG. 5, and the data tables in FIGS. 17-18.

Overall, MedDr consistently outperforms other GFMs across all datasets. Specifically, on the VQA-RAD dataset, MedDr achieves a 59.62% BLEU-1 score (P=0.062) and a 61.10% F1 score (P=0.054), much better than the fine-tuned LLaVA-Med. On the Slake-VQA dataset, MedDr obtains an 83.38% accuracy on close-ended questions and a 77.26% recall, surpassing RadFM by a large margin (P<0.001, subplot a of FIG. 5). For the most challenging dataset PMC-VQA, MedDr achieves a 27.30% recall and a 14.94% accuracy on open questions, outperforming other models significantly (P<0.001, the data table in FIG. 17). On the OmniMedVQA datasets, as illustrated in subplot b of FIG. 5 and the data table in FIG. 18, MedDr also establishes superior performance across all medical modalities, resulting in 63.0% overall accuracy. The overall performance of MedDr in the visual question answering task demonstrates that the proposed model not only excels in comprehending both visual and textual information but also handles a greater diversity of medical modalities.

The results of MRG tasks are presented in subplot c of FIG. 5 and the data table in FIG. 21. It should be noted that aside from RadFM [ref. 14] and MedDr, other models such as LLaVA-Med [ref. 12], Med-Flamingo [ref. 13], and InternVL [ref. 9] have not been trained on datasets of MRG tasks. MedDr outperforms RadFM [ref. 14] (overall, P≤0.001), which mainly focuses on radiology tasks, across nearly all evaluated metrics on both benchmark datasets. For example, MedDr achieves a ROUGE-L score of 22.59 on the MIMIC-CXR dataset and 28.35 on the IU-Xray dataset, highlighting its proficiency in comprehensively interpreting medical images.

FIG. 5, subplot d, is an example of medical report generation tasks on chest X-ray images. On the benchmark dataset MIMIC-CXR [ref. 21], MedDr is compared with RadFM [ref. 14], which is a GFM specialized in radiology. Most findings generated by RadFM only describe the normal (healthy) status of the patient, while abnormal information is more critical for medical reports. In contrast, MedDr lists both normal (e.g., “There is no evidence of pulmonary edema” and “There is no pneumothorax or large pleural effusion”) and abnormal findings (e.g., “There is increased opacity at the right lung base, which may represent atelectasis or early pneumonia.”) of the patient.

### GSCo Enables Accurate Disease Diagnosis and Generalizable AI for Medicine

In this section, experiments are conducted on medical image diagnosis datasets to demonstrate the effectiveness of the proposed GSCo framework. Following a previous study [ref. 51], ten representative models in computer vision are selected (e.g., ResNet [ref. 52] and ViT [ref. 53]) as the foundation (specialist) models, which possess significantly fewer parameters compared to the GFM. Details regarding the selected models may be found in the section below. These foundation models are fine-tuned on each of the 20 medical image diagnosis datasets, resulting in a total of 200 specialist models. Moreover, for a fair comparison, a baseline method, “Voting”, is introduced, where results are aggregated and voted from the predictions of the specialists, effectively functioning as a naive collaborative method to exploit the results of the specialist models.

### Quantitative Analysis

Subplots a-c of FIG. 6 illustrate the ranking order of various methods on the in-domain datasets and out-of-domain datasets, respectively. The summations of their rankings across different tasks are also presented herein. Firstly, it is observed that MedDr outperforms other GFMs and even surpasses some specialist models on in-domain datasets. Compared with specialist models, the overall performance of GFMs is lower, with most GFMs ranking relatively poorly. This observation indicates that while GFMs demonstrate superior generalizability through performing diverse tasks with a unified model, specialist models excel in precision on specific datasets due to their domain-specific fine-tuning. Notably, on in-domain datasets, MedDr showcases superior performance compared to several specialist models. For instance, on the ChestMNIST and PCam200 datasets, MedDr surpasses the majority of specialized models, achieving 4th and 5th place, respectively, which highlights its exceptional intrinsic diagnostic capabilities.

Secondly, it is found that GSCo achieves the highest overall performance, significantly surpassing other methods. As a straightforward collaborative method, “Voting” shows improvements on most datasets when compared with specialist models, highlighting its effectiveness. However, “Voting” obtains superior performance on binary classification and multi-class classification datasets while acquiring inferior results on multi-label datasets. This discrepancy suggests that it may struggle to effectively leverage the prediction results of specialist models on multi-label classification tasks. This is because, as a naive method, “Voting” relies solely on the outputs of the specialist models and treats their suggestions equally. Consequently, when confronted with highly challenging multi-label classification tasks, if the majority of specialized models provide incorrect predictions, “Voting” may yield erroneous diagnoses. In contrast, the proposed GSCo framework not only considers the predictions from the specialist models but also leverages the inherent knowledge of MedDr, leading to superior performance and robustness.

FIG. 6, subplot c, compares the specialist model with the best overall performance, DenseNet-121, against the proposed GSCo framework. For clarity, the results are categorized into three groups based on their classification task types. Compared with the best specialist model, GSCo exhibits a notable performance advantage, even on out-of-domain datasets, underscoring its superiority and generalizability.

Hence, the proposed GSCo framework exemplifies a synergistic relationship between GFM and specialist models. Specialist models, with their domain-specific knowledge, provide guidance to the GFM, thereby significantly enhancing its performance, especially on out-of-domain datasets. Additionally, fine-tuning the specialist models on specific downstream datasets is computationally efficient and may be implemented with minimal additional resources. On the other hand, the GFM acts as decision-makers with extensive intrinsic medical knowledge. Different from the “Voting” method, which relies solely on the outputs of specialist models, MedDr retains its diagnostic capabilities while integrating the guidance from these specialists. This collaborative strategy effectively bolsters performance across a wide range of medical tasks.

### Qualitative Analysis

The experimental results of mixture-of-expert diagnosis (MoED) and retrieval-augmented diagnosis (RAD) are visualized. Subplots a-c of FIG. 7, and FIG. 12 showcase examples of the MoED on downstream datasets across various medical modalities. Firstly, it is found that the aggregation of predictions from multiple specialist models yields accurate and robust guidance. In medical image diagnosis, the key to accurately identifying a disease often lies in the subtle nuances present within the image. Due to this inherent difficulty, among all specialist models, only EfficientNet-B4 [ref. 54] consistently produces correct diagnostic results across all example cases. Meanwhile, the aggregation of predictions from multiple specialist models, such as “Voting”, can derive more accurate results. For instance, on the RetOCT dataset (FIG. 7, subplot a) and CBIS-DDSM (CALC) dataset (FIG. 7, subplot b), the majority of specialist models make correct predictions, thereby providing MedDr with effective guidance to derive accurate diagnoses. Secondly, it is noted that even if the aggregating results fail to offer correct reference, MedDr can arrive at the correct diagnoses as well. For example, on the FMC-Colon dataset (FIG. 7, subplot c), half of the specialist models predict “Positive”, while the other half provide opposite results. While the naive “Voting” strategy fails in such a dilemma, MedDr generates the correct result “Negative”. These observations validate the superiority of MedDr and the efficacy of the proposed MoED. MedDr not only leverages the reference diagnoses provided by the specialist models but also utilizes its inherent knowledge to make the final decision. MoED represents an effective collaboration that integrates the strengths of both the Generalist and specialist models, thereby enhancing diagnostic accuracy.

Subplots d-f of FIG. 7, and FIG. 13 depict examples of the RAD on downstream datasets covering a broad range of modalities. Firstly, the effectiveness of the retrieval strategy is discussed. As illustrated in subplot d of FIG. 7 and FIG. 13e, the retrieved images share the same label as the query image, thereby serving as reliable references. Notably, although the specialist may render an incorrect diagnosis as a predictor, the majority of images retrieved still provide accurate diagnostic information, demonstrating the specialist's robust capability as a retriever. For instance, in subplot e of FIG. 7, the specialist model predicts the “Malignant” while most of the retrieved samples are “Normal, Benign”. These observations suggest that, in most cases, the retrieved samples are able to offer accurate guidance for MedDr, thereby validating the precision and robustness of the retrieval strategy. Secondly, the efficacy of RAD is discussed. In most cases of subplots d-f of FIG. 7, and FIG. 13, the retrieved samples provide correct guidance. Meanwhile, it is observed that even if the predictions from the specialist model and the retrieved items contain distracting information, MedDr can still make an accurate diagnosis based on its inherent disease diagnosis capability. For example, as shown in subplot f of FIG. 7, while most retrieved images are “Pneumonia” and therefore provide erroneous guidance, MedDr successfully derive the “Normal” diagnosis. These findings underscore that MedDr adeptly harnesses not only the external knowledge provided by the retrieved samples but also its inherent diagnostic capabilities, thereby demonstrating the effectiveness of the proposed RAD approach.

### Ablation Study

In this section, the ablation study of the proposed MoED and RAD mechanisms is discussed. Experiments are performed on nine medical image diagnosis datasets encompassing various medical modalities and tasks. FIG. 20 depicts the experimental results. For binary classification, accuracy is reported. Otherwise, the Macro-F1 score is reported. Both MoED and RAD demonstrate consistent performance improvements for MedDr, achieving average enhancements of 0.2030 and 0.2065, respectively. Compared with MoED, RAD exhibits superior performance, which can be attributed to its utilization of not only domain-specific knowledge from specialists but also information from the training database, thereby offering MedDr more reliable references.

To further assess the effectiveness and generalizability of RAD, experiments are conducted on broader types of downstream tasks. In visual question answering and medical report generation tasks, developing a specialist model that consistently outperforms a GFM can be particularly challenging. If specialist models underperform, they may fail to offer reliable guidance to the GFM, which could lead to a decline in overall performance. Therefore, in the following experiments, the vision encoder of MedDr as the retriever is adopted. For medical image diagnosis tasks, following previous practice, the labels of the five most similar cases are retrieved. In visual question answering and medical report generation tasks, the most similar images are retrieved and their corresponding annotations are then incorporated into the input. Additionally, Med-Flamingo [ref. 13] is also introduced as a baseline model, which showcases impressive few-show learning ability.

The data field in FIG. 22 depicts the results of medical image diagnosis, visual question answering, and medical report generation tasks. The “voting” column reflects the outcomes derived from a voting mechanism based on the labels of the retrieved samples. For medical report generation, the top-1 retrieved report is taken as the “voting” results. Notably, both Med-Flamingo and MedDr exhibit significant and consistent improvements across most datasets and tasks, underscoring the generalizability of RAD. However, Med-Flamingo's performance is upper-bounded by “voting”, indicating its heavy reliance on retrieved results for final diagnoses without adequately considering the image content. For instance, in the medical report generation task, Med-Flamingo often tends to directly rephrase or even replicate the retrieved samples with minimal modification. In contrast, MedDr consistently surpasses the “Voting” results across most downstream datasets, indicating that MedDr not only considers the retrieved results but also leverages its intrinsic knowledge for diagnosing the test images. Experiments validate the effectiveness of RAD, which can still enhance the capabilities of the GFM even in the absence of specialists, thereby boarding its application scenarios.

### Discussion

The proposed GSCo framework is the first piece of work to investigate the synergy between the GFM and specialist models. The GSCo framework is proposed to leverage the generalist's in-context learning abilities alongside the specialists' domain-specific knowledge. GSCo consists of two stages, namely the construction of GFM and specialists, and collaborative inference on downstream tasks. In the construction stage, MedDr, the largest open-source GFM tailored for medicine, is first developed to be capable of handling a wide range of medical tasks and modalities. MedDr also exhibits remarkable proficiency in both instruction-following and in-context learning, providing a solid foundation for cooperation with specialist models. Meanwhile, a series of lightweight specialists (models) are tailored for specific downstream tasks with low computational overhead. In the collaborative inference stage, Mixture-of-Expert Diagnosis (MoED) and Retrieval-Augmented Diagnosis (RAD) are proposed as the core mechanisms of the cooperation. MoED integrates predictions from the specialists as reference diagnoses, while RAD employs these specialists to retrieve similar cases, collectively providing MedDr with in-context information to facilitate medical image analysis. To evaluate MedDr and GSCo, the largest benchmark in medical GFM is curated, which consists of 28 datasets and about 250K testing samples, encompassing diverse medical modalities and tasks. Extensive qualitative and quantitative experiments highlight the following perspectives of the study.

It is found that MedDr excels in understanding and analysis. Compared with conventional models [ref. 9, 12-15], MedDr exhibits significant advantages in two key aspects. Firstly, as a generalist foundation model, MedDr demonstrates remarkable generalizability in medical image analysis, enabling it to process a broader scope of medical modalities, including radiology, pathology, dermatology, ophthalmology, and gastroenterology, and achieving state-of-the-art performance across various tasks, such as visual question answering, medical report generation, and medical image diagnosis. Secondly, MedDr showcases exceptional capabilities in-instruction following and in-context learning. This enhances the model's flexibility in handling different tasks and leveraging external knowledge, providing a solid foundation for effective collaboration with specialist models. These advantages can be attributed to the meticulously curated training corpus and the larger scale of MedDr. Concretely, the training corpus incorporates over 2 million samples across five distinct task types, thereby broadening the scope of MedDr. Additionally, with 40 billion parameters, MedDr surpasses previous models, endowing it with superior inherent capabilities. In the future, it is planned to incorporate more diverse training corpora as well as a larger foundation model.

It is further found that instruction following and in-context learning enables the Generalist-Specialist Collaboration. In most conventional models [ref. 9, 12-15, 26, 43], both GFMs and specialist models independently handle the downstream tasks with their inherent capabilities. GFMs are renowned for their generalizability and flexibility, enabling them to perform a variety of medical tasks across different modalities using a single model. In contrast, specialist models are esteemed for their precision and efficiency, achieving satisfactory performance by tailoring them to specific downstream tasks with low computational consumption. The proposed GSCo framework is devised to explore the collaboration between generalist and specialist models, wherein instruction following and in-context learning, which previous methods overlooked, act as essential links that facilitate their integration. Experimental results demonstrate that with exceptional instruction following and in-context learning capabilities, MedDr can effectively collaborate with specialist models and achieve SOTA performance on downstream tasks.

The GSCo framework presents a novel paradigm in the clinical application of GFM and specialists. In clinical practice, collaboration among healthcare professionals is not only common but also essential. The proposed GSCo framework illustrates that through the synergistic collaboration between the GFM and specialist models, superior performance on downstream tasks is achievable. GSCo presents a new paradigm in the clinical application of both GFM and specialists. Specifically, when confronted with out-of-domain tasks or data, rather than investing substantial resources to fine-tune the GFM, it is able to efficiently adapt lightweight specialist models with minimal resource expenditure. Additionally, due to the stringent privacy regulations governing most medical data, including Protected Health Information (PHI), directly fine-tuning the GFM on data from multiple sources is often impractical. Instead, under GSCo, the knowledge from these private datasets may be integrated by training the separate specialist models within their institutions independently, thereby safeguarding the confidentiality of medical data.

It is found that mixture-of-expert diagnosis and retrieval-augmented diagnosis provide effective collaboration strategies between the generalist and specialist models. For the study, two mechanisms are disclosed to provide MedDr with the guidance of the specialists, namely mixture-of-expert diagnosis and retrieval-augmented diagnosis. Mixture-of-expert diagnosis leverages the specialist model's predictions as references, while retrieval-augmented diagnosis treats the specialist model as a retriever to access relevant information in the training dataset. In such collaboration, the roles of MedDr and specialists are distinct. The specialist model plays a pivotal role in equipping the GFM with reference guidance to enhance its generalization capabilities, particularly for out-of-domain tasks. Meanwhile, MedDr functions as a decision-maker with a wealth of medical knowledge. Through extensive experiments, it is demonstrated that these mechanisms enable the GFM to benefit from the specialist model's expertise, resulting in a robust and generalizable AI in the field of medicine/healthcare.

The limitations and further future directions of the proposed framework are discussed. Despite the advancements, the experimental results also reveal some limitations, providing clear directions for future enhancements and research. Firstly, while GSCo has demonstrated satisfactory performance across various public datasets, further exploration of its application in clinical practice is necessary to substantiate the superiority of the methodology. Secondly, multimodal retrieval strategies should be further explored. GSCo showcases the significant potential of retrieval-augmented generation in medical image analysis. However, the current vision-based retrieval method still struggles to ensure the accuracy of the retrieved samples. Lastly, diverse collaborative paradigms require further exploration. While GSCo has validated the effectiveness of collaboration, it is anticipated observing more interaction patterns between generalists and specialists. For instance, incorporating Chain of Thought (CoT) [ref. 55] could be a viable approach to facilitating collaboration between generalists and specialists.

### Method

### Construction of GFM and Specialists

### Diagnosis-Guided Bootstrapping and Medical Image Description Dataset

The proposed Diagnosis-Guided Bootstrapping (DGB) and Medical Image Description (DES) datasets are discussed herein, in accordance with various aspects of the disclosure. To leverage the abundant medical image diagnosis datasets, the DGB dataset is first presented. Conventional solutions [ref. 12, 14] have constructed instruction-tuning datasets primarily derived from PubMed research articles, to alleviate the scarcity of vision-language data in the medical domain. Although these strategies successfully assembled large-scale training corpora, they present two significant limitations. Firstly, they rely solely on textual information, neglecting visual elements, which may lead to inconsistencies in descriptions. Secondly, the content of research articles might lack reliability and accuracy, thereby introducing noise into the training corpus.

In contrast, in accordance with aspects of the disclosure, it is proposed to generate the instruction tuning dataset based on medical image diagnosis datasets, exploiting multi-modal information and human-verified annotations. As shown in subplot a of FIG. 9, it is observed that GFMs in the general domain [ref. 8, 9] exhibit a comprehensive understanding of disease-related information and associated symptoms, owing to extensive training on diverse corpora. Nonetheless, these models encounter difficulties in correlating this knowledge with specific medical images, leading to inaccurate diagnostic predictions. Conversely, when provided with specific disease and modality information alongside the given image, the GFM in the general domain becomes capable of generating high-quality medical reports with accurate diagnoses. For example, subplot b of FIG. 9 depicts a generated report on “ulcerative colitis”, where findings enumerate the observations in the image and the impression encapsulates the conclusion.

Motivated by the above observations, it is proposed to devise a diagnosis-guided bootstrapping strategy that leverages both visual and textual information to construct the instruction-tuning dataset. Specifically, the instruction may be formatted as shown in FIG. 8, but not to be limited as such. The model is provided with information about the modality and disease of the medical image and is required to generate a detailed report.

In contrast to conventional solutions [ref. 12, 14], which generated data from textual information only, the proposed approach provides two prominent advantages. Firstly, it facilitates the utilization of numerous human-verified label-level annotated datasets in medicine. Secondly, incorporating both visual and textual information ensures the generated information remains pertinent to the accompanying images. Following this method, the DGB dataset encompassing diverse medical image modalities is built.

Meanwhile, to augment the diversity of the training data (for MedDr), the Medical Image Description (DES) datasets are provided. Specifically, image-based case studies are collected from OpenI [ref. 36] and the GFM [ref. 9] is employed to write a description of the image from a medical perspective, integrating both the image and associated textual information. Additionally, details that are not inferable directly from the image, such as the patient's name or age, are excluded to maintain focus on diagnostically relevant visual features.

### Medical Instruction Tuning

To enhance the various capabilities of the GFM, following conventional solutions [ref. 12-14], visual question answering [ref. 14, 19, 37, 47, 56] and medical report generation [ref. 21, 22] datasets are incorporated into the training corpus. Additionally, to augment the diversity of the data, image-based case studies are also collected from OpenI [ref. 36]. Overall, as depicted in FIG. 1, subplot b, the training corpus encompasses five different types of items: medical image diagnosis (CLS), medical report generation (MRG), visual question answering (VQA), diagnosis-guided bootstrapping (DGB), and medical image description (DES). It is proposed to meticulously craft the prompt template for each type, as depicted listed in FIG. 25. More detailed information about the training datasets is presented in the section below. The language modeling loss is utilized as the loss function to train the model.

### Collaborative Inference on Downstream Tasks

### Mixture-of-Expert Diagnosis

When addressing a specific downstream task, training a lightweight specialist model may more often be practical than utilizing GFMs. This advantage is primarily due to the significantly lower training overhead while still achieving satisfactory performance. In this study, the collaborative potential between generalist and specialist models is investigated, to enhance outcomes on downstream tasks.

As shown in subplots a and b of FIG. 10, several lightweight models are selected and trained on the downstream dataset, designating these as specialist models. Unlike the GFM, these specialist models are tailored to specific downstream tasks, yielding superior performance on these tasks due to expert knowledge. During the inference phase, the testing image is first input into the specialist models and their predictions are utilized as a guiding reference, which are subsequently incorporated into the instruction. MedDr integrates both the testing image and the reference predictions to render the final diagnosis.

### Retrieval-Augmented Diagnosis

GFMs have demonstrated significant capabilities but still face challenges when encountering out-of-domain data [ref. 9, 57]. To exploit the training corpus exhaustively and enhance the capability on out-of-domain datasets, a collaborative mechanism, Retrieval-Augmented Diagnosis, is further proposed to assist MedDr.

Subplots a-c of FIG. 11 illustrate the proposed collaborative mechanism, where a database is built on the training data across multiple medical tasks and modalities. To build the database, given an image-text pair, the image is encoded by an embedding model and this visual embedding is arranged to be the key while the text is arranged to be the value. So each image is associated with its visual embedding. During inference, the embedding model is employed to encode the test image. Then, the derived visual embedding of the test image is used to query the database by calculating the vector (e.g. cosine) similarity between the query and keys in the database. The top-k (k is a positive integer) most similar items from the database are retrieved, and their meta information is incorporated into the instruction as additional textual clues to help the model make medical decisions.

For this study, utilizing the specialist model as the retriever is first explored. After fine-tuning on the specific downstream dataset, this specialist model is capable of generating more discriminative embeddings, thereby enhancing retrieval accuracy. It is to be appreciated that in various examples, the best specialist model (from all selected specialist models employed for RAG) may function to perform the role of the retriever. Furthermore, application of the vision encoder from MedDr as the retriever is explored, which may provide two advantages. First, it effectively eliminates the need for an additional embedding module, which in turn reduces associated computational costs. By leveraging the vision encoder from MedDr as the embedding module, the intermediate results may directly be used as embeddings for queries (at the database) during inference without incurring any extra overhead. Second, this approach is applicable to a wider array of scenarios, particularly in situations where acquiring a qualified specialist model poses challenges.

### Implementation Details

While not to be construed as limiting, InternVL [ref. 9], a state-of-the-art GFM in the general domain, may be selected and used as the foundation model for MedDr, which contains about 40 billion parameters, consisting of a 6 billion vision encoder and a 34 billion large language model. The input image is resized to 448×448 pixels. The model is fine-tuned on both collected and generated data. The number of training samples is about 2 million. Please refer to the foregoing discussions for detailed information about the training dataset and instruction prompt. The instruction tuning recipe follows the suggestions provided by InternVL. All parameters are fixed except for the LoRA component, which is composed of approximately 0.1 billion parameters, accounting for 0.4% of the total parameters. Meanwhile, DeepSpeed ZeRO Stage 3 [ref. 58] is also leveraged to optimize the training procedure. The model is trained on 16 NVIDIA H800 GPUs for two epochs within 72 hours. The specialist models are trained on a single NVIDIA 4090 GPU and the corresponding recipe is depicted by the data field in FIG. 28.

### State-of-the-Art Generalist and Specialist Models

In this study, 4 open-source models in both the general and medical domains are selected as the baseline GFMs.

RadFM [ref. 14] mainly focuses on the radiology modality. It consists of a 3D ViT as the vision backbone and PMC-LLaMA-13B [ref. 59] as the LLM. The model is first trained on 16 million noisy pre-training data and then fine-tuned on 3 million in-domain data.

LLaVA-Med [ref. 12] is built on the pre-trained LLaVA [ref. 8]. It is fine-tuned on about 600K concept alignment samples and 60K instruction tuning samples in one day with 8 A100 GPUS.

Med-Flamingo [ref. 13] is developed based on OpenFlamingo-9B [ref. 60], which can handle multiple images interleaved with texts. It is trained on large-scale interleaved datasets based on medical textbooks and the PMC-OA dataset [ref. 61].

InternVL [ref. 9] is one of the most powerful open-source large-scale vision-language models in the general domain. It obtains SOTA performance on multi-modal tasks in the general domain.

For these GFMs, the above models are reproduced based on their open-source checkpoint and the models are evaluated using the same test data. The test prompt is set up according to their official implementations (see the data table in FIG. 31).

For the choice of the specialist models, following [ref. 51], ten representative computer vision models are selected and trained on the specific downstream datasets:
VGG16 [ref. 62] is a convolution-based model. It consists of 16 layers and is known for its simplicity and effectiveness.
AlexNet [ref. 63] is a convolution-based model. It won the ImageNet Large Scale Visual Recognition Challenge (ILSVRC) in 2012 and popularized the use of convolutional neural networks (CNNs).
ResNet-18 [ref. 52] is a convolution-based model. It introduced residual connections that allow gradients to flow directly through the network, enabling the training of very deep networks.
DenseNet-121 [ref. 64] is a convolution-based model. It encourages feature reuse by connecting each layer to every other layer in a feed-forward fashion.
EfficientNet-B4 [ref. 54] is a convolution-based model. It balances depth, width, and resolution to achieve high performance with fewer parameters. It is efficient in terms of both accuracy and computation.
ViT-B/16 [ref. 53] is a Transformer-based model. It applies the Transformer architecture to image data, achieving competitive results on various vision tasks.
CLIP ViT-B/16 [ref. 4] is a Transformer-based model. It learns to associate images and text in a joint embedding space, enabling zero-shot classification and other multimodal tasks.
EVA-02 ViT-B/16 [ref. 65] is a Transformer-based model. It improves the training techniques for CLIP at scale and achieves superior performance with significantly smaller training costs.
DINO ViT-B/16 [ref. 66] is a Transformer-based model. It leverages clustering and momentum encoders and achieves strong performance without using labeled data.
SAM ViT-B/16 [ref. 67] is a Transformer-based model, which is designed to be a general-purpose model for image segmentation, capable of segmenting any object in an image with minimal user input.

The details of these specialist models are listed in FIG. 27. Particularly, the specifications of the ten selected computer vision models are detailed, encompassing the number of parameters, Giga Multiply-Add Operations per Second (GMACs), and the training time per epoch on a dataset, with PneumoniaMNIST serving as an example. Parameters are quantified in millions (M), and “IN1K” refers to “ImageNet-1K”.

The fine-tuning recipe on the downstream dataset is shown in FIG. 28, where hyperparameters used for training each vision model are listed. The unique parameters include input dimension, hidden dimension, and dropout rate. The common parameters are batch size, number of epochs, optimizer, learning rate, scheduler, and weight decay.

Compared with the GFMs, these specialist models have much fewer parameters, and may be trained on consumer-level hardware, such as NVIDIA 4090 GPU.

### Training Dataset

The training dataset is introduced in detail herein and FIG. 25 depicts the prompt template for each type of dataset.

### Visual Question Answering Datasets

SLAKE [ref. 47] is a bilingual radiology VQA dataset comprising 642 images and 14K questions. Only the English part of the training split is used, which consists of 4919 question-answer pairs.

VQA-RAD [ref. 37] is a manually constructed dataset where clinicians asked naturally occurring questions of radiology images and provided reference answers. Following the official split, 3064 question-answer pairs of the training set are used.

Path-VQA [ref. 19] consists of 32799 open-ended questions from 4998 pathology images, where each question is manually checked to ensure correctness. Following the official split, 19755 question-answer pairs of the training set are used.

PMC-VQA [ref. 56] is a large-scale medical visual question-answering dataset built from image-text pairs from PubMed Central, covering broader medical image modalities. Following the official split, 152603 question-answer pairs of the training set are used.

PMC-CaseReport [ref. 14] is an auto-generated visual question-answering dataset based on the case report papers in the PMC-Inline dataset. Following the official split, 254105 question-answer pairs of the training set are used.

### Medical Report Generation Datasets

MIMIC-CXR [ref. 21] presents 371,920 chest X-rays associated with 227943 imaging studies from 65079 patients. Following RadFM [ref. 14] and R2Gen [ref. 68], 337292 cases are used for training.

IU-Xray [ref. 22] is a set of chest X-ray images paired with their corresponding diagnostic reports. The dataset contains 7470 pairs of images and reports. Following R2Gen [ref. 68], 4730 cases from the training split are used.

### Medical Image Diagnosis Datasets

VinDr-SpineXR [ref. 25] is a large annotated medical image dataset for spinal lesion detection and classification from radiographs. Following RadFM [ref. 68], 6129 samples are used for training.

VinDr-PCXR [ref. 30] is an open-source large-scale pediatric chest X-ray dataset for the interpretation of common thoracic diseases. Following RadFM [ref. 68], 4585 samples are used for training.

VinDr-Mammo [ref. 69] is a large-scale benchmark dataset for computer-aided detection and diagnosis in full-field digital mammography. Following RadFM [ref. 68], 6047 samples are used for training.

VinDr-CXR [ref. 70] is an open large-scale dataset of chest X-rays with radiologist's annotations. The training set contains 15000 scans, and 3 radiologists independently label each image. Following the official split, 45000 samples are used for training.

CheXpert [ref. 71] is a large public dataset for chest radiograph interpretation, consisting of 224316 chest radiographs of 65240 patients. Following the official split, 223414 samples are used for training.

ChestX-ray14 [ref. 33] is a medical imaging dataset which comprises 112120 frontal-view X-ray images of 30805 patients with the text-mined 14 common disease labels. Following the official split, 86524 samples are used for training.

PCam200 [ref. 72] is a public pathological H&E image dataset made in the same manner from Camelyon2016 challenge dataset [ref. 73]. Following the official split, 28539 samples are used for training.

PAD-UFES-20 [ref. 34] is a dermatology classification dataset consisting of 2298 images for six different diagnostics. All the 2298 samples are used for training.

DermNet [ref. 44] consists of dermatology images of 23 types of skin diseases taken from DermNet. Following the official split, 15557 samples are used for training.

HAM10000 [ref. 31] is a large collection of multi-source dermatoscopic images of pigmented lesions. Following the official split, 10015 samples are used for training.

ISIC2020 [ref. 74] is a dataset of the SIIM-ISIC Melanoma Classification Challenge 2020. The dataset contains 33126 dermoscopic training images of unique benign and malignant skin lesions from over 2000 patients. Following the official split, 33126 samples are used for training.

Kvasir [ref. 75] is a multi-class image dataset for computer-aided gastrointestinal disease detection. Following the official split, 8000 samples are used for training.

Kvasir Capsule [ref. 35] is an endoscopy dataset consisting of 47238 images with annotations of anatomical landmarks and pathological and normal findings. Following the official split, 47238 samples are used for training.

WCE [ref. 76] is a curated colon disease dataset based on Kvasir [ref. 75] and ETIS-Larib-Polyp DB [ref. 77]. Following the official split, 3200 samples are used for training.

GastroVision [ref. 78] is a multi-center open-access gastrointestinal (GI) endoscopy dataset that includes different anatomical landmarks, pathological abnormalities, polyp removal cases, and normal findings from the GI tract. All the 8000 samples are used for training.

ODIR [ref. 79] is a structured ophthalmic database of 5000 patients with age, color fundus photographs from left and right eyes, and doctors' diagnostic keywords. Following the official split, 6392 samples are used for training.

Fundus1000 [ref. 80] contains 1000 fundus images with 39 categories. All the 1000 samples are used for training.

RFMiD2.0 [ref. 32] is a multi-label dataset including around 860 retinal fundus images annotated by three eye specialists. Following the official split, 455 samples are used for training.

Retinal OCT-C8 [ref. 45] is a large-scale dataset for ophthalmic research containing 24000 optical coherence tomography (OCT) images that are organized into eight categories. Following the official split, 18000 samples are used for training.

UltraBreast is a private breast ultrasound dataset that contains 45896 cases that are labeled benign or malignant.

### Synthetic Datasets

Diagnosis-Guided Bootstrapping Dataset is part of the synthetic datasets. As afore discussed, a large-scale medical report dataset across diverse medical modalities is constructed. Specifically, 196760 samples in total are generated based on the VinDr-SpineXR [ref. 25], VinDr-PCXR [ref. 30], VinDr-Mammo [ref. 69], VinDr-CXR [ref. 70], ChestX-ray 14 [ref. 33], PADUFES-20 [ref. 34], Dermnet [ref. 44], Kvasir [ref. 75], WCE [ref. 76], Kvasir Capsule [ref. 35], ODIR [ref. 79], Fundus1000 [ref. 80] and RFMiD2.0 [ref. 32] datasets.

Medical Image Description dataset is considered to be part of the synthetic datasets. To augment the diversity of the training data, 245371 image-based case studies are collected from OpenI [ref. 36]. The title of the case and the image caption are summarized based on the image by InternVL [ref. 9], and high-quality images and corresponding text summaries are obtained.

### Out-of-Domain Benchmark Dataset

### Visual Question Answering Datasets

VQA-Med [ref. 20] focuses on radiology images and consists of four main categories of questions: modality, plane, organ system, and abnormality. Following the official split, 500 items are used for testing.

OmniMedVQA [ref. 28] is a large-scale comprehensive evaluation benchmark dataset for the medical GFMs. Due to an overlap with the training data, some data are excluded to prevent data leakage and only the Disease Diagnosis subset are used. The total number of the testing samples is 51977.

### Medical Image Diagnosis Datasets

PneumoniaMNIST [ref. 26] is a binary classification dataset about chest X-ray. Following the official split, 624 items are used for testing.

BreastMNIST [ref. 26] is a binary classification dataset of breast ultrasound. Following the official split, 156 samples are used for testing.

OrganAMNIST [ref. 26] is a multi-class classification dataset of abdominal CT. Following the official split, 17778 items are used for testing.

PathMNIST [ref. 26] is a multi-class classification dataset of colon pathology. Following the official split, 7180 items are used for testing.

OCTMNIST [ref. 26] a multi-class classification dataset of retinal OCT. Following the official split, 1000 items are used for testing.

ChestMNIST [ref. 26] is a multi-label dataset of chest X-ray. Following the official split, 22433 samples are used for testing.

CBIS-DDSM [ref. 50] contains images for screening mammography. The original dataset contains images of cases with three conditions of breast cancer: BENIGN, BENIGN WITHOUT CALLBACK, and MALIGNANT. Due to the insufficient information in the text to discriminate between BENIGN and BENIGN WITHOUT CALLBACK, it is formulated as a binary classification task. Following the official split, 326 samples are used from the CALC subset and 378 samples are used from MASS for testing.

FMC-Colon [ref. 27] is a pathological tumor tissue classification dataset and requests the model to determine whether the sample is positive or negative. Following the official split, 4355 samples are used for testing.

FMC-Endo [ref. 27] is a colonoscopy lesion classification dataset and consists of four different lesion types. Following the official split, 2055 samples are used for testing.

FMC-Chest [ref. 27] is a thoracic disease screening dataset and covers 19 common thoracic abnormalities. Following the official split, 2708 samples are used for testing.

Derm7pt [ref. 81] is a dataset for evaluating computerized image-based prediction of the 7-point skin lesion malignancy checklist. Following the official split, 395 samples are used for testing.

BRSET [ref. 46] is a multi-labeled ophthalmological dataset designed to improve scientific community development and validate machine learning models. The dataset is randomly divided into training and testing splits with an 8:2 ratio. The number of testing samples is 3254.

### Evaluation Metrics

### Medical Image Diagnosis

For the medical image diagnosis datasets, accuracy and F1-Score are exploited for evaluation. Accuracy is calculated based on equation (1):

$$Accuracy = \frac{1}{N} {\sum }_{i}^{N} 1 ( {y}_{i} = {\hat{y}}_{l} ) \qquad ( 1 )$$

wherein y is a tensor of target values, and ŷ is a tensor of predictions. F1-Score is defined based on recall and precision, following from equations (2)-(4):

$$Recall = \frac{TP}{TP + FN} \qquad ( 2 )$$

$$Precision = \frac{TP}{TP + FP} \qquad ( 3 )$$

$$F 1-S core = 2 \cdot \frac{Precision * Recall}{Precision + Recall} \qquad ( 4 )$$

wherein TP and FP represent the number of true positives and false positives respectively. Especially, for multi-class and multi-label classification datasets, both Macro-F1 score and micro-F1 score are calculated.

### Visual Question Answering

For the dataset consisting of multiple-choice questions [ref. 28], the accuracy is calculated. For other visual question answering datasets, following MultiMedEval [ref. 49], tokenization of both prediction and answer is first performed, and precision and recall is then computed. For close-ended questions, the prediction is correct if its recall is at least 0.5. For open-ended questions, the prediction is correct if its recall is at least 0.5. For open-ended questions, the prediction is correct if its recall is at least 0.75. The accuracy of closed-ended questions and the accuracy of open-ended questions is reported. Additionally, following [ref. 14], the BLEU-1 score is computed from equation (5):

$$BLEU - 1 = BP \cdot exp ( {\sum }_{n = 1}^{N} {w}_{n} \cdot {log p}_{n} ) \qquad ( 5 )$$

wherein BP is the brevity penalty, p is the precision for n-grams, and wₙ is the weight for n-gram precision. If the predicting result's length c is greater than the reference length r, then BP=1. If c≤r, then BP=exp(1−r/c). This ensures that a shorter predicting result is penalized to prevent the system from favoring overly concise output. Since there is only one type of n-gram, w₁=1.

### Medical Report Generation

For medical report generation tasks, there is utilization of common n-gram-based metrics such as BLEU-1, BLEU-4, ROUGE-1, ROUGE-L, and METEOR [ref. 82]. Here, ROUGE-1 is defined as per equation (6):

$$ROUG \text{E-1} = \frac{\vert Recall ⋂ Reference \vert}{\vert Reference \vert} \qquad ( 6 )$$

wherein |Recall ∩ Reference| is the number of overlapping unigrams between the generated report and the reference report, whereas |Reference| refers to the total number of unigrams in the reference text. ROUGE-L is defined based on equation (7):

$$ROUG \text{E-} L = \frac{{F}_{LCS}}{\vert Reference \vert} \qquad ( 7 )$$

wherein F_{LCS} represents the F1 score of the longest common subsequence. The METEOR score is computed in accordance with equation (8):

$$METEOR = \frac{1}{m} {\sum }_{gϵ gold} {max}_{h ∈ hyp} Precision ( g , h ) \qquad ( 8 )$$

wherein m is the number of gold standard (reference) sentences, and Precision (g, h) refers to the precision score between a specific gold standard (g) and a hypothesis sentence (h) form the set of all gold standard sentences (gold) and the set of all hypotheses sentences (hyp).

Moreover, F1-RadGraph is evaluated, which measures the F1 score between entities extracted from the reference and generated reports using RadGraph [ref. 83] based on equation (9):

$$F 1- R adGraph = 2 \cdot \frac{Precision \cdot Recall}{Precision + Recall} \qquad ( 9 )$$

The CheXbert vector similarity [ref. 84] is also computed using cosine similarity between the embedded reference and generated reports as per equation (10):

$$Cosine Similarity = \frac{A \cdot B}{‖ A ‖ ‖ B ‖} \qquad ( 10 )$$

wherein A and B are the vectors of the reference and generated reports, respectively.

### Data Availability

The datasets used for building the training dataset are listed in FIG. 29, and the evaluation benchmark datasets are listed in FIG. 30. Regarding the respective labels at the “Access” column in FIGS. 29-30, it is to be noted that “Open Access” datasets are freely available to the public, while for the “Restricted Access” datasets, the respective dataset providers are to be contacted for access permissions. “Credentialed Access” datasets require specific permissions, and “Private” datasets are not publicly accessible.

FIG. 32 is a block diagram of a training manager 3205 for training a generalist foundation model (GFM), in accordance with aspects of the present disclosure. The training manager 3205 may implement in software vis-à-vis the method of FIG. 2 described herein. The training manager 3205 may be executed by a computer device 3400, 3500, as depicted in FIGS. 34-35, or its components. The training manager 3205 may include a generating component 3210, a configuring component 3215, and a training component 3220. Each of these components may communicate 3225, directly or indirectly, with one another (e.g. via one or more buses).

The generating component 3210 may cause to generate, by the GFM to be provided as a first dataset, a plurality of medical reports related to different medical conditions, wherein each medical report is generated based on an associated image showing a medical condition, and classification labels that include textual description about the medical condition, and a medical modality associated with the medical condition.

The generating component 3210 may cause to further generate, by the GFM, textual description for respective images showing different medical conditions, wherein the textual description describe the medical conditions, based on diagnostically relevant visual features shown in the respective images, and wherein the generated textual description and the respective images are provided as a second dataset.

The configuring component 3215 may configure a training dataset to include the first dataset, the second dataset, a third dataset associated with Medical Image Diagnosis (CLS), a fourth dataset associated with Medical Report Generation (MRG), and a fifth dataset associated with Visual Question Answering (VQA).

The training component 3220 may train, based on the training dataset, the GFM to obtain a medically-trained GFM.

Alternatively, each of the above described components 3210, 3215, 3220 in the training manager 3205 (including itself) may be realized as specific hardware modules (e.g. ASICs) to perform those same operations. Notwithstanding, implementation of said components 3210, 3215, 3220 may optionally be realized via a mix of hardware and software modules, as desired.

FIG. 33 is a block diagram of a coordination manager 3305 for processing a sample image of a medical condition, in accordance with aspects of the present disclosure. The coordination manager 3305 may be an implementation in software for the method of FIG. 3 described herein. The coordination manager 3305 may be executed by a computer device 3400, 3500, as depicted in FIGS. 34-35, or its components. The coordination manager 3305 may include a processing component 3310, and a querying component 3315. Each of these components may communicate 3320, directly or indirectly, with one another (e.g. via one or more buses).

The processing component 3310 may cause to process, by a set of computer vision models for a medical modality associated with the medical condition, the sample image to obtain a set of determination related to a plurality of predicted diagnoses for the medical condition, wherein the set of computer vision models are selected from a plurality of different sets of computer vision models, wherein respective sets of computer vison models are pretrained with downstream datasets specific to respective medical modalities for different medical condition.

The processing component 3310 may cause to process, by an embedding model, the sample image to determine an associated visual embedding to be used as a query for querying a database, wherein the database include a plurality of entries of value-key pairs associated with respective images of different medical conditions; and wherein a key represents a visual embedding of an image, and an associated value provides textual description of the medical condition shown in said image.

The querying component 3315 may query, based on vector similarity between the determined visual embedding and the respective keys in the plurality of the entries, the database to retrieve a top-k most similar entries from the database, wherein k is a positive integer.

The processing component 3310 may cause to process, by a medically-trained generalist foundation model (GFM) in conjunction with using the set of determination related to the plurality of predicted diagnoses as reference context and images corresponding with the retrieved top-k most similar entries, the sample image to make a predicted determination on the medical condition shown by the sample image, wherein the medically-trained GFM is trained in accordance with the method 200 discussed under FIG. 2. To clarify, the medically-trained GFM refers to the proposed MedDr.

Alternatively, each of the above described components 3310, 3315 in the coordination manager 3305 (including itself) may be realized as specific hardware modules (e.g. ASICs) to perform those same operations. Yet further, implementation of said components 3310, 3315 may optionally be realized via a combination of hardware and software modules, as desired.

FIG. 34 is a schematic diagram of an exemplary (first) computing device 3400 that may be utilized for performing the method 200 of FIG. 2 or the method 300 of FIG. 3, in accordance with aspects of the present disclosure.

The computing device 3400 comprises a keypad 3402, a touch-screen 3404, a microphone 3406, a speaker 3408 and an antenna 3410. The computing device 3400 may be operated by a user to perform a variety of different functions/tasks, for example, hosting a telephone call, sending an SMS message, browsing the Internet, sending emails and/or providing satellite navigation.

The computing device 3400 comprises hardware to perform communication functions (e.g. telephony, or data communication), together with an application processor and corresponding support hardware to enable the computing device 3400 to have other functions, for example, messaging, Internet browsing, email functions or the like. The communication hardware includes a radio frequency (RF) processor 3412 which provides an RF signal to the antenna 3410 for the transmission of data signals, and the receipt therefrom. Additionally provided is a baseband processor 3414, which provides signals to and receives signals from the RF processor 3412. The baseband processor 3414 may also interact with a subscriber identity module (SIM) 3416, as known in the art. The communication subsystem enables the computing device 3400 to communicate via a number of different communication protocols including 3G, 4G, 5G, New Radio (NR), GSM, WiFi, Bluetooth™ and/or CDMA. The communication subsystem of the computing device 3400 is beyond the scope of the present invention.

The keypad 3402 and the touch-screen 3404 are controlled by an application processor 3418. A power and audio controller 3420 is provided to supply power from a battery 3422 to the communication subsystem, the application processor 3418, and the other hardware. The power and audio controller 3420 also controls input from the microphone 3406, and audio output via the speaker 3408. Also provided is a global positioning system (GPS) antenna and associated receiver element 3424, which is controlled by the application processor 3418 and is capable of receiving a GPS signal for use with a satellite navigation functionality of the computing device 3400.

Various different types of memory may be provided in the computing device 3400 to supplement the operations of the application processor 3418. The computing device 3400 may include Random Access Memory (RAM) 3426 coupled to the application processor 3418 into which data and program code may be written and read from. Code stored in RAM 3426 may be executed by the application processor 3418 from RAM 3426. RAM 3426 represents a form of volatile memory of the computing device 3400.

The computing device 3400 may further be provided with a non-volatile (long-term) storage 3428 coupled to the application processor 3418. The storage 3428 may logically be divided into three partitions being an operating system (OS) partition 3430, a system partition 3432 and a user partition 3434. The storage 3428 may represent a non-volatile memory of the computing device 3400.

In the present example, the OS partition 3430 may include firmware of the computing device 3400, which includes an operating system. Other computer programs may also be stored on the storage 3428, such as application programs (also referred to as apps), and the like. In particular, application programs considered critical for operations of the computing device 3400, for example, in the case of a smartphone, communications applications and the like, may typically be stored in system partition 3432. The application programs stored on the system partition 3432 typically may be programmed in the computing device 3400 in its factory setting.

Application programs subsequently added and installed on the computing device 3400 by the user may typically be stored in the user partition 3434.

The various functional components illustrated in FIG. 34 may alternatively be collocated into a single component. For example, the storage 3428 may comprise NAND flash, NOR flash, a hard disk drive or a combination of these.

FIG. 35 is a schematic diagram of an exemplary (second) computing device 3500 that may be utilized for performing the method 200 of FIG. 2 or the method 300 of FIG. 3, in accordance with aspects of the present disclosure. The following description of the computing device 3500 is provided by way of example only and is not intended to be limiting.

As depicted in FIG. 35, the example computing device 3500 includes a processor 3504 for executing software routines. Although a single processor is shown for the sake of clarity, the computing device 3500 may also include a multi-processor system. The processor 3504 is connected to a communication infrastructure 3506 for communication with other components of the computing device 3500. The communication infrastructure 3506 may include, for example, a communications bus, a crossbar network, or a network.

The computing device 3500 further includes a main memory 3508, such as a random-access memory (RAM), and a secondary memory 3510. The secondary memory 3510 may include, for example, a hard disk drive 3512 and/or a removable storage drive 3514, which may include a floppy disk drive, a magnetic tape drive, an optical disk drive, or the like. The removable storage drive 3514 reads from and/or writes to a removable storage unit 3518, as known in the art. The removable storage unit 3518 may include a floppy disk, magnetic tape, optical disk, or the like, which is read by and written to by removable storage drive 1314. As may be appreciated in the art, the removable storage unit 3518 includes a computer readable storage medium having stored therein computer executable program code instructions and/or data.

In other aspects, the secondary memory 3510 may additionally or alternatively include other similar means for allowing computer programs or other instructions to be loaded into the computing device 3500. Such means may include, for example, a removable storage unit 3522 and an associated interface 3520. Examples of a removable storage unit 3522 and interface 3520 may include a program cartridge and cartridge interface (e.g. such as that found in video game console devices), a removable memory chip (such as an EPROM or PROM) and associated socket, and other exemplary removable storage units 3522 and interfaces 3520, which may enable software programs and/or data to be transferred between the removable storage unit 3522 and the computer system 3500.

The computing device 3500 also includes at least one communication interface 3524. The communication interface 3524 allows software programs and data to be transferred between computing device 3500 and external devices via a communication path 3526. In various aspects, the communication interface 3524 permits data to be transferred between the computing device 3500 and a data communication network, such as a public data or private data communication network. The communication interface 3524 may be used to exchange data between different computing devices 3500 that may together form part of an interconnected computer network. Examples of a communication interface 3524 may include a modem, a network interface (e.g. an Ethernet card), a communication port, an antenna with associated circuitry or the like. The communication interface 3524 may be wired or may be wireless. Software and data transferred via the communication interface 3524 are in the form of signals which can be electronic, electromagnetic, optical or other signals capable of being received by communication interface 3524. These signals are provided to the communication interface via the communication path 3526.

The computing device 3500 further includes a display interface 3502 which is configured to perform operations for rendering images to an associated display 3530 and an audio interface 3532 for performing operations for playing audio content via associated speaker(s) 3534.

As used herein, the term “computer program product” may refer, in part, to the removable storage unit 3518, the removable storage unit 3522, a hard disk installed in the hard disk drive 3512, or a carrier wave carrying software over the communication path 3526 (wireless link or cable) to the communication interface 3524. Computer readable storage media refers to any non-transitory tangible storage medium that provides recorded instructions and/or data to the computing device 3500 for execution and/or processing. Examples of such storage media include floppy disks, magnetic tape, CD-ROM, DVD, Blu-Ray™ Disc, a hard disk drive, a ROM or integrated circuit, USB memory, a magneto-optical disk, or a computer readable card such as a PCMCIA card or the like, whether or not such devices are internal or external of the computing device 3500. Examples of transitory or non-tangible computer readable transmission media that may also participate in the provision of software, application programs, instructions and/or data to the computing device 3500 include radio or infra-red transmission channels as well as a network connection to another computer or networked device, and the Internet or Intranets including e-mail transmissions and information recorded on Websites and the like.

The computer programs (also termed computer program code/instruction) are stored in the main memory 3508 and/or the secondary memory 3510. Computer programs may also be received via the communication interface 3524. Such computer programs, when executed, enable the computing device 3500 to perform one or more aspects of the present disclosure afore discussed. In various aspects of the present disclosure, the computer programs, which when executed, enable the processor 3504 to perform aspect(s) of the present disclosure. Accordingly, such computer programs may represent controllers of the computer system 3500.

Software may be stored in a computer program product and loaded into the computing device 3500 using the removable storage drive 3514, the hard disk drive 3512, or the interface 3520. Alternatively, the computer program product may be downloaded to the computer system 3500 over the communication path 3526. The software, when executed by the processor 3504, causes the computing device 3500 to perform aspects of the present disclosure.

It is to be understood that the computing device 3500 in FIG. 35 is presented merely by way of example. Hence, in some aspects, one or more features of the computing device 3500 may be omitted. Also, in other aspects, one or more features of the computing device 3500 may be combined together, or collocated. Additionally, in some aspects, one or more features of the computing device 3500 may be divided into one or more component parts.

It is to be appreciated that the elements illustrated in FIG. 35 may function to provide means for performing the various functions of the method 200 of FIG. 2 or the method 300 of FIG. 3, as described in accordance with aspects of the present disclosure. Also, the term “computing device” 3400, 3500 may include or may be referred to as a mobile device, a wireless device, a remote device, a handheld device, a tablet computer, a laptop computer, a computer server, a computer terminal, a blade server, among other examples. The computer device 3400, 3500 described herein may be able to communicate with various types of devices, such as other computer devices 3400, 3500 that may sometimes act as relays, or work together under configuration to function as a computer cluster for performing high-performance computing.

All of the methods described herein describe possible implementations, and that the operations and the steps may be rearranged or otherwise modified and that other implementations are possible. Further, aspects from two or more of the methods, if applicable, may be combined.

Information and signals described herein may be represented using any of a variety of different technologies and techniques. For example, data, instructions, commands, information, signals, bits, symbols, and chips that may be referenced throughout the description may be represented by voltages, currents, electromagnetic waves, magnetic fields or particles, optical fields or particles, or any combination thereof.

The various illustrative blocks and components described in connection with the disclosure herein may be implemented or performed with a general-purpose processor, a DSP, an ASIC, a CPU, an FPGA or other programmable logic device, discrete gate or transistor logic, discrete hardware components, or any combination thereof designed to perform the functions described herein. A general-purpose processor may be a microprocessor, but in the alternative, the processor may be any processor, controller, microcontroller, or state machine. A processor may also be implemented as a combination of computing devices (for example, a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described herein may be implemented using software executed by a processor, hardware, firmware, hardwiring, or combinations of any of these. Features implementing functions may also be physically located at various positions, including being distributed such that portions of functions are implemented at different physical locations.

Computer-readable media includes both non-transitory computer storage media and communication media including any medium that facilitates transfer of a computer program from one place to another. A non-transitory storage medium may be any available medium that may be accessed by a general-purpose or special purpose computer. By way of example, and not limitation, non-transitory computer-readable media may include RAM, ROM, electrically erasable programmable ROM (EEPROM), flash memory, compact disk (CD) ROM or other optical disk storage, magnetic disk storage or other magnetic storage devices, or any other non-transitory medium that may be used to carry or store desired program code means in the form of instructions or data structures and that may be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of computer-readable medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

As used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (such as, A and B and C). Also, as used herein, the phrase “based on” shall not be construed as a reference to a closed set of conditions. For example, an example step that is described as “based on condition A” may be based on both a condition A and a condition B without departing from the scope of the present disclosure. In other words, as used herein, the phrase “based on” shall be construed in the same manner as the phrase “based at least in part on”.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label, or other subsequent reference label.

The description set forth herein, in connection with the appended drawings, describes example configurations and does not represent all the examples that may be implemented or that are within the scope of the claims. The term “example” used herein means “serving as an example, instance, or illustration,” and not “preferred” or “advantageous over other examples”. The detailed description includes specific details for the purpose of providing an understanding of the described techniques. These techniques, however, may be practiced without these specific details. In some instances, known structures and devices are shown in block diagram form in order to avoid obscuring the concepts of the described examples.

The description herein is provided to enable a person having ordinary skill in the art to make or use the disclosure. Various modifications to the disclosure will be apparent to a person having ordinary skill in the art, and the generic principles defined herein may be applied to other variations without departing from the scope of the disclosure. Thus, the disclosure is not limited to the examples and designs described herein, but to be accorded the broadest scope consistent with the principles and novel features disclosed herein.

### Examples

The following examples are disclosed, in accordance with aspects of the present disclosure.
Example 1: A computer-implemented method for training a generalist foundation model (GFM), the method comprises: generating, by the GFM to be provided as a first dataset, a plurality of medical reports related to different medical conditions, wherein each medical report is generated based on an associated image showing a medical condition, and classification labels that include textual description about the medical condition, and a medical modality associated with the medical condition; generating, by the GFM, textual description for respective images showing different medical conditions, wherein the textual description describe the medical conditions, based on diagnostically relevant visual features shown in the respective images, and wherein the generated textual description and the respective images are provided as a second dataset; configuring a training dataset to include the first dataset, the second dataset, a third dataset associated with Medical Image Diagnosis (CLS), a fourth dataset associated with Medical Report Generation (MRG), and a fifth dataset associated with Visual Question Answering (VQA); and training, based on the training dataset, the GFM to obtain a medically-trained GFM.
Example 2: The method of example 1, wherein the GFM includes being selected from one of: RadFM, LLaVA-Med, Med-Flamingo, and Intern VL.
Example 3: The method of example 1, wherein each medical report is configured to include, based on processing by the GFM, information of medical findings and impressions about the medical condition shown by the associated image.
Example 4: The method of example 1, wherein the respective images showing the different medical conditions are obtained from OpenI.
Example 5: The method of example 1, wherein the medical modality includes one of: radiology, pathology, dermatology, ophthalmology, gastroenterology, fundoscopy, chest X-ray, and endoscopy.
Example 6: A computer-implemented method for processing a sample image of a medical condition, the method comprises: processing, by a set of computer vision models for a medical modality associated with the medical condition, the sample image to obtain a set of determination related to a plurality of predicted diagnoses for the medical condition, wherein the set of computer vision models are selected from a plurality of different sets of computer vision models, wherein respective sets of computer vision models are pretrained with downstream datasets specific to respective medical modalities for different medical conditions; processing, by an embedding model, the sample image to determine an associated visual embedding to be used as a query for querying a database, wherein the database include a plurality of entries of value-key pairs associated with respective images showing different medical conditions; and wherein a key represents a visual embedding of an image, and an associated value provides textual description of the medical condition shown in said image; querying, by at least one computer vision model in the selected set of computer vision models based on vector similarity between the determined visual embedding and the respective keys in the plurality of the entries, the database to retrieve a top-k most similar entries from the database, wherein k is a positive integer; and processing, by a medically-trained generalist foundation model (GFM) using the obtained set of determination as reference context and images corresponding with the retrieved top-k most similar entries, the sample image to make a predicted determination on the medical condition shown by the sample image, wherein the medically-trained GFM is trained in accordance with the method of example 1.
Example 7: The method of example 6, wherein the vector similarity is performed based on cosine similarity.
Example 8: A computing device for training a generalist foundation model (GFM), comprising: one or more memories having executable code; and one or more processors coupled to the one or more memories, and configured to execute the code to cause the device to: generate, by the GFM to be provided as a first dataset, a plurality of medical reports related to different medical conditions, wherein each medical report is generated based on an associated image showing a medical condition, and classification labels that include textual description about the medical condition, and a medical modality associated with the medical condition; generate, by the GFM, textual description for respective images showing different medical conditions, wherein the textual description describe the medical conditions, based on diagnostically relevant visual features shown in the respective images, and wherein the generated textual description and the respective images are provided as a second dataset; configure a training dataset to include the first dataset, the second dataset, a third dataset associated with Medical Image Diagnosis (CLS), a fourth dataset associated with Medical Report Generation (MRG), and a fifth dataset associated with Visual Question Answering (VQA); and train, based on the training dataset, the GFM to obtain a medically-trained GFM.
Example 9: The device of example 8, wherein the GFM includes being selected from one of: RadFM, LLaVA-Med, Med-Flamingo, and Intern VL.
Example 10: The device of example 8, wherein the respective images showing the different medical conditions are obtained from OpenI.
Example 11: The device of example 8, wherein the medical modality includes one of: radiology, pathology, dermatology, ophthalmology, gastroenterology, fundoscopy, chest X-ray, and endoscopy.
Example 12: A computing device for processing a sample image of a medical condition, comprising: one or more memories having executable code; and one or more processors coupled to the one or more memories, and configured to execute the code to cause the device to: process, by a set of computer vision models for a medical modality associated with the medical condition, the sample image to obtain a set of determination related to a plurality of predicted diagnoses for the medical condition, wherein the set of computer vision models are selected from a plurality of different sets of computer vision models, wherein respective sets of computer vision models are pretrained with downstream datasets specific to respective medical modalities for different medical conditions; process, by an embedding model, the sample image to determine an associated visual embedding to be used as a query for querying a database, wherein the database include a plurality of entries of value-key pairs associated with respective images showing different medical conditions; and wherein a key represents a visual embedding of an image, and an associated value provides textual description of the medical condition shown in said image; query, by at least one computer vision model in the selected set of computer vision models based on vector similarity between the determined visual embedding and the respective keys in the plurality of the entries, the database to retrieve a top-k most similar entries from the database, wherein k is a positive integer; and process, by a medically-trained generalist foundation model (GFM) using the obtained set of determination as reference context and images corresponding with the retrieved top-k most similar entries, the sample image to make a predicted determination on the medical condition shown by the sample image, wherein the medically-trained GFM is trained in accordance with the method of example 1.
Example 13: The device of example 12, wherein the vector similarity is performed based on cosine similarity.
Example 14: A non-transitory computer-readable medium comprising executable code, which when executed by a processor of a computing device, cause the device to perform the method of any of examples 1-5.
Example 15: A non-transitory computer-readable medium comprising executable code, which when executed by a processor of a computing device, cause the device to perform the method of any of examples 6-7.
Example 16: A computing device for training a generalist foundation model (GFM), comprising: means for generating, by the GFM to be provided as a first dataset, a plurality of medical reports related to different medical conditions, wherein each medical report is generated based on an associated image showing a medical condition, and classification labels that include textual description about the medical condition, and a medical modality associated with the medical condition; means for generating, by the GFM, textual description for respective images showing different medical conditions, wherein the textual description describe the medical conditions, based on diagnostically relevant visual features shown in the respective images, and wherein the generated textual description and the respective images are provided as a second dataset; means for configuring a training dataset to include the first dataset, the second dataset, a third dataset associated with Medical Image Diagnosis (CLS), a fourth dataset associated with Medical Report Generation (MRG), and a fifth dataset associated with Visual Question Answering (VQA); and means for training, based on the training dataset, the GFM to obtain a medically-trained GFM.
Example 17: A computing device for processing a sample image of a medical condition, comprising: means for processing, by a set of computer vision models for a medical modality associated with the medical condition, the sample image to obtain a set of determination related to a plurality of predicted diagnoses for the medical condition, wherein the set of computer vision models are selected from a plurality of different sets of computer vision models, wherein respective sets of computer vision models are pretrained with downstream datasets specific to respective medical modalities for different medical conditions; means for processing, by an embedding model, the sample image to determine an associated visual embedding to be used as a query for querying a database, wherein the database include a plurality of entries of value-key pairs associated with respective images showing different medical conditions; and wherein a key represents a visual embedding of an image, and an associated value provides textual description of the medical condition shown in said image; means for querying, by at least one computer vision model in the selected set of computer vision models based on vector similarity between the determined visual embedding and the respective keys in the plurality of the entries, the database to retrieve a top-k most similar entries from the database, wherein k is a positive integer; and means for processing, by a medically-trained generalist foundation model (GFM) using the obtained set of determination as reference context and images corresponding with the retrieved top-k most similar entries, the sample image to make a predicted determination on the medical condition shown by the sample image, wherein the medically-trained GFM is trained in accordance with the method of example 1.

### REFERENCES

[1] Achiam, J. et al. Gpt-4 technical report. arXiv preprint arXiv: 2303.08774 (2023).
[2] Touvron, H. et al. Llama 2: Open foundation and fine-tuned chat models. arXiv preprint arXiv: 2307.09288 (2023).
[3] Anil, R. et al. Palm 2 technical report. arXiv preprint arXiv: 2305.10403 (2023).
[4] Radford, A. et al. Learning transferable visual models from natural language supervision, 8748-8763 (PMLR, 2021).
[5] Tung, C., Lin, Y., Yin, J., Ye, Q. & Chen, H. Exploring vision language pretraining with knowledge enhancement via large language model, 81-91 (Springer, 2024).
[6] Xu, Y. et al. A multimodal knowledge-enhanced whole-slide pathology foundation model. arXiv preprint arXiv: 2407.15362 (2024).
[7] Zhu, D., Chen, J., Shen, X., Li, X. & Elhoseiny, M. Minigpt-4: Enhancing vision-language understanding with advanced large language models. arXiv preprint arXiv: 2304.10592 (2023).
[8] Liu, H., Li, C., Wu, Q. & Lee, Y. J. Visual instruction tuning. Advances in neural information processing systems 36 (2024).
[9] Chen, Z. et al. Interval: Scaling up vision foundation models and aligning for generic visual-linguistic tasks. arXiv preprint arXiv: 2312.14238 (2023).
[10] Antol, S. et al. Vqa: Visual question answering, 2425-2433 (2015).
[11] Lin, T.-Y. et al. Microsoft coco: Common objects in context, 740-755 (Springer, 2014).
[12] Li, C. et al. Llava-med: Training a large language-and-vision assistant for biomedicine in one day. Advances in Neural Information Processing Systems 36 (2024).
[13] Moor, M. et al. Med-flamingo: a multimodal medical few-shot learner, 353-367 (PMLR, 2023).
[14] Wu, C., Zhang, X., Zhang, Y., Wang, Y. & Xie, W. Towards generalist foundation model for radiology. arXiv preprint arXiv: 2308.02463 (2023).
[15] Tu, T. et al. Towards generalist biomedical ai. NEJM AI 1, AIoa2300138 (2024).
[16] Moor, M. et al. Foundation models for generalist medical artificial intelligence. Nature 616, 259-265 (2023).
[17] Saab, K. et al. Capabilities of gemini models in medicine. arXiv preprint arXiv: 2404.18416 (2024).
[18] Yang, L. et al. Advancing multimodal medical capabilities of gemini. arXiv preprint arXiv: 2405.03162 (2024).
[19] He, X., Zhang, Y., Mou, L., Xing, E. & Xie, P. Pathvqa: 30000+ questions for medical visual question answering. arXiv preprint arXiv: 2003.10286 (2020).
[20] Ben Abacha, A. et al. Vqa-med: Overview of the medical visual question answering task at imageclef 2019, Vol. 2380 of CEUR Workshop Proceedings (CEUR-WS.org, Lugano, Switzerland, 2019). URL: https://ceur-ws.org/Vol-2380/pape r 272.pdf.
[21] Johnson, A. E. et al. Mimic-cxr, a de-identified publicly available database of chest radiographs with free-text reports. Scientific data 6, 317 (2019).
[22] Demner-Fushman, D. et al. Preparing a collection of radiology examinations for distribution and retrieval. Journal of the American Medical Informatics Association 23, 304-310 (2016).
[23] Jin, H., Che, H., Lin, Y. & Chen, H. Promptmrg: Diagnosis-driven prompts for medical report generation, Vol. 38, 2607-2615 (2024).
[24] Chen, Z., Luo, L., Bie, Y. & Chen, H. Dia-llama: Towards large language model-driven ct report generation. arXiv preprint arXiv: 2403.16386 (2024).
[25] Nguyen, H. T. et al. Vindr-spinexr: A deep learning framework for spinal lesions detection and classification from radiographs, 291-301 (Springer, 2021).
[26] Yang, J. et al. Medmnist v2-a large-scale lightweight benchmark for 2d and 3d biomedical image classification. Scientific Data 10, 41 (2023).
[27] Wang, D. et al. A real-world dataset and benchmark for foundation model adaptation in medical image classification. Scientific Data 10, 574 (2023).
[28] Hu, Y. et al. Omnimedvqa: A new large-scale comprehensive evaluation benchmark for medical lvlm. arXiv preprint arXiv: 2402.09181 (2024).
[29] Chen, P. et al. Gmai-mmbench: A comprehensive multimodal evaluation benchmark towards general medical ai. arXiv preprint arXiv: 2408.03361 (2024).
[30] Pham, H. H., Tran, T. T. & Nguyen, H. Q. Vindr-pcxr: An open, large-scale pediatric chest x-ray dataset for interpretation of common thoracic diseases. PhysioNet (version 1.0.0) 10 (2022).
[31] Tschandl, P., Rosendahl, C. & Kittler, H. The ham10000 dataset, a large collection of multi-source dermatoscopic images of common pigmented skin lesions. Scientific data 5, 1-9 (2018).
[32] Panchal, S. et al. Retinal fundus multi-disease image dataset (rfmid) 2.0: A dataset of frequently and rarely identified diseases. Data 8 (2023). URL https://www.mdpi.com/2306-5729/8/2/29.
[33] Wang, X. et al. Chestx-ray8: Hospital-scale chest x-ray database and benchmarks on weakly-supervised classification and localization of common thorax diseases, 2097-2106 (2017).
[34] Pacheco, A. G. et al. Pad-ufes-20: A skin lesion dataset composed of patient data and clinical images collected from smartphones. Data in brief 32, 106221 (2020).
[35] Smedsrud, P. H. et al. Kvasir-Capsule, a video capsule endoscopy dataset. Scientific Data 8, 142 (2021).
[36] Demner-Fushman, D., Antani, S., Simpson, M. & Thoma, G. R. Design and development of a multimodal biomedical information retrieval system. Journal of Computing Science and Engineering 6, 168-177 (2012).
[37] Lau, J. J., Gayen, S., Demner, D. & Abacha, A. B. Visual question answering in radiology (vqa-rad). Open Science Framework (2018).
[38] Jacobs, R. A., Jordan, M. I., Nowlan, S. J. & Hinton, G. E. Adaptive mixtures of local experts. Neural computation 3, 79-87 (1991).
[39] Fedus, W., Zoph, B. & Shazeer, N. Switch transformers: Scaling to trillion parameter models with simple and efficient sparsity. Journal of Machine Learning Research 23, 1-39 (2022).
[40] Luo, L. et al. Towards non-invasive and personalized management of breast cancer patients from multiparametric mri via a large mixture-of-modality-experts model. arXiv preprint arXiv: 2408.12606 (2024).
[41] Xiong, C. et al. Mome: Mixture of multimodal experts for cancer survival prediction. arXiv preprint arXiv: 2406.09696 (2024).
[42] Lewis, P. et al. Retrieval-augmented generation for knowledge-intensive nlp tasks. Advances in Neural Information Processing Systems 33, 9459-9474 (2020).
[43] Zhang, K. et al. A generalist vision-language foundation model for diverse biomedical tasks. Nature Medicine 1-13 (2024).
[44] Goel, S. Dermnet. https://www.kaggle.com/datasets/shubhamgoel27/dermnet (2020).
[45] Subramanian, M., Shanmugavadivel, K., Naren, O. S., Premkumar, K. & Rankish, K. Classification of retinal oct images using deep learning, 1-7 (2022)
[46] Nakayama, L. F. et al. A brazilian multilabel ophthalmological dataset (brset). PhysioNet https://doi. org/10 13026 (2023).
[47] Liu, B. et al. Slake: A semantically-labeled knowledge-enhanced dataset for medical visual question answering, 1650-1654 (IEEE, 2021).
[48] Zhang, X. et al. Pmc-vqa: Visual instruction tuning for medical visual question answering. arXiv preprint arXiv: 2305.10415 (2023).
[49] Royer, C., Menze, B. & Sekuboyina, A. Multimedeval: A benchmark and a toolkit for evaluating medical vision-language models (2024). 2402.09262.
[50] Sawyer-Lee, R., Gimenez, F., Hoogi, A. & Rubin, D. Curated breast imaging sub-set of digital database for screening mammography (cbis-ddsm) [skup podataka]. The cancer imaging archive (2016).
[51] Doerrich, S., Di Salvo, F., Brockmann, J. & Ledig, C. Rethinking model prototyping through the medmnist+ dataset collection. arXiv preprint arXiv: 2404.15786 (2024).
[52] He, K., Zhang, X., Ren, S. & Sun, J. Deep residual learning for image recognition, 770-778 (2016).
[53] Dosovitskiy, A. et al. An image is worth 16×16 words: Transformers for image recognition at scale. arXiv preprint arXiv: 2010.11929 (2020).
[54] Tan, M. & Le, Q. Efficientnet: Rethinking model scaling for convolutional neural networks, 6105-6114 (PMLR, 2019).
[55] Wei, J. et al. Chain-of-thought prompting elicits reasoning in large language models. Advances in neural information processing systems 35, 24824-24837 (2022).
[56] Zhang, X. et al. Pmc-vqa: Visual instruction tuning for medical visual question answering. arXiv preprint arXiv: 2305.10415 (2023).
[57] Van Veen, D. et al. Radadapt: Radiology report summarization via lightweight domain adaptation of large language models. arXiv preprint arXiv: 2305.01146 (2023).
[58] Rajbhandari, S., Ruwase, O., Rasley, J., Smith, S. & He, Y. Zero-infinity: Breaking the gpu memory wall for extreme scale deep learning, 1-14 (2021).
[59] Wu, C., Zhang, X., Zhang, Y., Wang, Y. & Xie, W. Pmc-llama: Further finetuning llama on medical papers. arXiv preprint arXiv: 2304.14454 (2023).
[60] Awadalla, A. et al. Openflamingo: An open-source framework for training large autoregressive vision-language models. arXiv preprint arXiv: 2308.01390 (2023).
[61] Lin, W. et al. Pmc-clip: Contrastive language-image pre-training using biomedical documents, 525-536 (Springer, 2023).
[62] Simonyan, K. & Zisserman, A. Very deep convolutional networks for large-scale image recognition. arXiv preprint arXiv: 1409.1556 (2014).
[63] Krizhevsky, A., Sutskever, I. & Hinton, G. E. Imagenet classification with deep convolutional neural networks. Advances in neural information processing systems 25 (2012).
Huang, G., Liu, Z., Van Der Maaten, L. & Weinberger, K. Q. Densely connected convolutional networks, 4700-4708 (2017).
[65] Fang, Y. et al. Eva-02: A visual representation for neon genesis. arXiv preprint arXiv: 2303.11331 (2023).
[66] Caron, M. et al. Emerging properties in self-supervised vision transformers, 9650-9660 (2021).
[67] Kirillov, A. et al. Segment anything, 4015-4026 (2023).
[68] Chen, Z., Song, Y., Chang, T.-H. & Wan, X. Generating radiology reports via memory-driven transformer (2020).
[69] Nguyen, H. T. et al. Vindr-mammo: A large-scale benchmark dataset for computer-aided diagnosis in full-field digital mammography. Scientific Data 10, 277 (2023).
[70] Nguyen, H. Q. et al. Vindr-cxr: An open dataset of chest x-rays with radiologist's annotations (2020). 2012.15029.
[71] Irvin, J. et al. Chexpert: A large chest radiograph dataset with uncertainty labels and expert comparison, Vol. 33, 590-597 (2019).
[72] Kawai, M., Ota, N. & Yamaoka, S. Large-scale pretraining on pathological images for fine-tuning of small pathological benchmarks, 257-267 (Springer, 2023).
[73] Bejnordi, B. E. et al. Diagnostic assessment of deep learning algorithms for detection of lymph node metastases in women with breast cancer. Jama 318, 2199-2210 (2017).
[74] Rotemberg, V. et al. A patient-centric dataset of images and metadata for identifying melanomas using clinical context. Scientific data 8, 34 (2021).
[75] Pogorelov, K. et al. Kvasir: A multi-class image dataset for computer aided gastrointestinal disease detection, 164-169 (2017).
[76] Montalbo, F. J. Wce curated colon disease dataset deep learning. https://www.ka ggle.com/datasets/francismon/curated-colon-dataset-for-deep-learning (2022).
[77] Silva, J., Histace, A., Romain, O., Dray, X. & Granado, B. Toward embedded detection of polyps in wce images for early diagnosis of colorectal cancer. International journal of computer assisted radiology and surgery 9, 283-293 (2014).
[78] Jha, D. et al. Gastrovision: A multi-class endoscopy image dataset for computer aided gastrointestinal disease detection, 125-140 (Springer, 2023).
[79] Li, N., Li, T., Hu, C., Wang, K. & Kang, H. A benchmark of ocular disease intelligent recognition: One shot for multi-disease detection, 177-193 (Springer, 2021).
[80] Cen, L.-P. et al. Automatic detection of 39 fundus diseases and conditions in retinal photographs using deep neural networks. Nature communications 12, 4828 (2021).
[81] Kawahara, J., Daneshvar, S., Argenziano, G. & Hamarneh, G. Seven-point check-list and skin lesion classification using multitask multimodal neural nets. IEEE journal of biomedical and health informatics 23, 538-546 (2018).
[82] Banerjee, S. & Lavie, A. Meteor: An automatic metric for mt evaluation with improved correlation with human judgments, 65-72 (2005).
[83] Jain, S. et al. Radgraph: Extracting clinical entities and relations from radiology reports.
[84] Yu, F. et al. Evaluating progress in automatic chest x-ray radiology report generation. Patterns 4 (2023).

What is claimed is:

1. A computer-implemented method for training a generalist foundation model (GFM), the method comprising:
generating, by the GFM to be provided as a first dataset, a plurality of medical reports related to different medical conditions, wherein each medical report is generated based on an associated image showing a medical condition, and classification labels that include textual description about the medical condition, and a medical modality associated with the medical condition;
generating, by the GFM, textual description for respective images showing different medical conditions, wherein the textual description describe the medical conditions, based on diagnostically relevant visual features shown in the respective images, and wherein the generated textual description and the respective images are provided as a second dataset;
configuring a training dataset to include the first dataset, the second dataset, a third dataset associated with Medical Image Diagnosis (CLS), a fourth dataset associated with Medical Report Generation (MRG), and a fifth dataset associated with Visual Question Answering (VQA); and
training, based on the training dataset, the GFM to obtain a medically-trained GFM.

2. The method of claim 1, wherein the GFM includes being selected from one of: RadFM, LLaVA-Med, Med-Flamingo, and Intern VL.

3. The method of claim 1, wherein each medical report is configured to include, based on processing by the GFM, information of medical findings and impressions about the medical condition shown by the associated image.

4. The method of claim 1, wherein the respective images showing the different medical conditions are obtained from OpenI.

5. The method of claim 1, wherein the medical modality includes one of: radiology, pathology, dermatology, ophthalmology, gastroenterology, fundoscopy, chest X-ray, and endoscopy.

6. A computer-implemented method for processing a sample image of a medical condition, the method comprising:
processing, by a set of computer vision models for a medical modality associated with the medical condition, the sample image to obtain a set of determination related to a plurality of predicted diagnoses for the medical condition, wherein the set of computer vision models are selected from a plurality of different sets of computer vision models, wherein respective sets of computer vision models are pretrained with downstream datasets specific to respective medical modalities for different medical conditions;
processing, by an embedding model, the sample image to determine an associated visual embedding to be used as a query for querying a database, wherein the database include a plurality of entries of value-key pairs associated with respective images of different medical conditions; and wherein a key represents a visual embedding of an image, and an associated value provides textual description of the medical condition shown in said image;
querying, by at least one computer vision model in the selected set of computer vision models based on vector similarity between the determined visual embedding and the respective keys in the plurality of the entries, the database to retrieve a top-k most similar entries from the database, wherein k is a positive integer; and
processing, by a medically-trained generalist foundation model (GFM) using the obtained set of determination as reference context and images corresponding with the retrieved top-k most similar entries, the sample image to make a predicted determination on the medical condition shown by the sample image,
wherein the medically-trained GFM is trained in accordance with the method of claim 1.

7. The method of claim 6, wherein the vector similarity is performed based on cosine similarity.

8. A computing device for training a generalist foundation model (GFM), comprising:
one or more memories having executable code; and
one or more processors coupled to the one or more memories, and configured to execute the code to cause the device to:
generate, by the GFM to be provided as a first dataset, a plurality of medical reports related to different medical conditions, wherein each medical report is generated based on an associated image showing a medical condition, and classification labels that include textual description about the medical condition, and a medical modality associated with the medical condition;
generate, by the GFM, textual description for respective images showing different medical conditions, wherein the textual description describe the medical conditions, based on diagnostically relevant visual features shown in the respective images, and wherein the generated textual description and the respective images are provided as a second dataset;
configure a training dataset to include the first dataset, the second dataset, a third dataset associated with Medical Image Diagnosis (CLS), a fourth dataset associated with Medical Report Generation (MRG), and a fifth dataset associated with Visual Question Answering (VQA); and
train, based on the training dataset, the GFM to obtain a medically-trained GFM.

9. The device of claim 8, wherein the GFM includes being selected from one of: RadFM, LLaVA-Med, Med-Flamingo, and Intern VL.

10. The device of claim 8, wherein the respective images showing the different medical conditions are obtained from OpenI.

11. The device of claim 8, wherein the medical modality includes one of: radiology, pathology, dermatology, ophthalmology, gastroenterology, fundoscopy, chest X-ray, and endoscopy.

12. A computing device for processing a sample image of a medical condition, comprising:
one or more memories having executable code; and
one or more processors coupled to the one or more memories, and configured to execute the code to cause the device to:
process, by a set of computer vision models for a medical modality associated with the medical condition, the sample image to obtain a set of determination related to a plurality of predicted diagnoses for the medical condition, wherein the set of computer vision models are selected from a plurality of different sets of computer vision models, wherein respective sets of computer vision models are pretrained with downstream datasets specific to respective medical modalities for different medical conditions;
process, by an embedding model, the sample image to determine an associated visual embedding to be used as a query for querying a database, wherein the database include a plurality of entries of value-key pairs associated with respective images of different medical conditions; and wherein a key represents a visual embedding of an image, and an associated value provides textual description of the medical condition shown in said image;
query, by at least one computer vision model in the selected set of computer vision models based on vector similarity between the determined visual embedding and the respective keys in the plurality of the entries, the database to retrieve a top-k most similar entries from the database, wherein k is a positive integer; and
process, by a medically-trained generalist foundation model (GFM) using the obtained set of determination as reference context and images corresponding with the retrieved top-k most similar entries, the sample image to make a predicted determination on the medical condition shown by the sample image,
wherein the medically-trained GFM is trained in accordance with the method of claim 1.

13. The device of claim 12, wherein the vector similarity is performed based on cosine similarity.

14. A non-transitory computer-readable medium comprising executable code, which when executed by a processor of a computing device, cause the device to perform the method of claim 1.

15. A non-transitory computer-readable medium comprising executable code, which when executed by a processor of a computing device, cause the device to perform the method of claim 6.
